(19) United States
(12) Patent Application Publication
(10) Pub. No.: US 2024/0315629 A1
(43) Pub. Date: Sep. 26, 2024

(54) CONDUCTIVE NANOMATERIALS AND COMPOSITES THEREOF

(71) Applicant: NORTHWESTERN UNIVERSITY, Evanston, IL (US)

(72) Inventors: Jonathan Rivnay, Chicago, IL (US); Joshua Tropp, Evanston, IL (US)

(21) Appl. No.: 18/613,535

(22) Filed: Mar. 22, 2024

Related U.S. Application Data
(60) Provisional application No. 63/491,753, filed on Mar. 23, 2023.

(51) Int. Cl. A61B 5/266; A61B 5/268; C08G 61/12; C08L 5/04; C08L 25/18; C08L 65/00

(52) U.S. Cl. CPC A61B 5/266; A61B 5/268; C08G 61/126; C08L 5/04; C08L 25/18; C08L 65/00; C08G 2261/3223; C08L 2201/06; C08L 2203/02; C08L 2203/20

(57) ABSTRACT

The invention in one aspect relates to a conductive nanomaterial comprising acid-crystalized poly(3,4-ethylenedioxythiophene) poly(styrenesulfonate) (PEDOT:PSS) nanoparticles (ncrys-PEDOT_{X}) with intrinsic dispersibility.

### CROSS-REFERENCE TO RELATED PATENT APPLICATION

This application claims priority to and the benefit of U.S. Provisional Application Ser. No. 63/491,753, filed Mar. 23, 2023, which is incorporated herein in its entirety by reference.

### STATEMENT AS TO RIGHTS UNDER FEDERALLY-SPONSORED RESEARCH

This invention was made with government support under grant number N00014-20-1-2777 awarded by the Department of Defense. The government has certain rights in the invention.

### FIELD OF THE INVENTION

The present invention relates generally to the material science, and more particularly to conductive nanomaterials including acid crystallized PEDOT particles and composites thereof.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Bioelectricity is vital to many physiological processes in living organisms, such as the contraction of muscles, transmission of nerve impulses, regulation of heartbeats, and tissue regeneration, among others. The desire to implement bioelectricity to monitor or influence biological events has led to the emergence of bioelectronics, a discipline which aims to merge biology and microelectronics-typically through implantable or wearable devices. These medical technologies require a high-quality interface between tissue and electronics that enables both ionic and electronic communication. The first-generation of bioelectronic devices relied on inorganic interfaces which pose several disadvantages including 1) a strain mismatch which can lead to cell death and fibrosis at the tissue-device interface, 2) poor compatibility with magnetic fields used during magnetic resonance imaging (MRI), 3) challenges associated with translating both ionic and electronic signals, and 4) limited biodegradability. To overcome these challenges, researchers have been developing soft organic materials with mixed ionic and electronic conduction that can seamlessly integrate with cells and living tissues. Conductive hydrogels have gained significant attention as their structure mimics the extracellular matrix, the natural microenvironment of the cells, as such conductive hydrogels, have been aggressively explored for tissue engineering and regenerative medicine (TERM). The biological basis of such TERM applications is the facilitated transmission of endogenous or exogenous bioelectricity throughout the hydrogel, requiring charge percolation throughout the entire material.

Conductive hydrogels include hydrophilic polymeric networks with incorporated electroactive fillers, the most common of which include carbon nanotubes, graphene nanomaterials, conjugated polymers, and inorganics (gold, silver, metal oxides, etc.). Rationally designing hydrogels to promote homogeneous, 3D charge percolation for TERM and other bioelectronic applications remains a challenge. A significant limitation is the surface energy mismatch between the hydrophilic hydrogel precursor and the hydrophobic, typically carbon-based nanomaterial filler; this incompatibility requires laborious, multistep procedures to achieve sufficient loading for charge percolation. Of the available conductive fillers, conjugated polymers (CPs) have distinct advantages due to their enhanced chemical tunability, flexibility, biocompatibility, and solution processability. To achieve the necessary intrachain and interchain charge transport across long length scales for sufficient conductivity in thin-films, there are many processing methods such as spin-coating, blade-coating, or other facile techniques to obtain favorable packing with minimal polymer. However, as these processing methods cannot be translated to the tortuous, porous form factor of a hydrogel, achieving this same favorable packing is challenging, and has been traditionally limited to incorporating high loadings of CP filler.

Alternatively, CPs can be preorganized into colloidal dispersions which are stabilized by polyelectrolytes surfactants—enabling both solution processibility and high electrical conductivity. The most prominent CP dispersion is poly(3,4-ethylenedioxythiophene) doped with poly(styrenesulfonate) (PEDOT:PSS) which can feature conductivities beyond 4000 S cm⁻¹ when processed appropriately, leading to its widespread adoption within thin film applications such as chemical sensing, energy storage, and antistatic coatings. The commercial presence and biocompatibility of PEDOT:PSS has also inspired its use within conductive hydrogels for biomedical applications; however, as the ink is designed for 2D coatings, its direct incorporation within 3D hydrogels typically affords low conductivities between 10⁻³ and 10⁻¹ S cm⁻¹ (Table 2). While several groups have formulated conductive hydrogels directly out of PEDOT:PSS dispersions, many applications require the chemical and mechanical features of commonplace biomaterials to facilitate biocompatibility and biological outcomes such as cellular differentiation, necessitating a method to endow natural and synthetic biomaterials with conductivity.

Therefore, a heretofore unaddressed need exists in the art to address the aforementioned deficiencies and inadequacies.

### SUMMARY OF THE INVENTION

In one aspect, the invention relates to a conductive nanomaterial comprising acid-crystalized poly(3,4-ethylenedioxythiophene) poly(styrenesulfonate) (PEDOT:PSS) nanoparticles (ncrys-PEDOT_{X}) with intrinsic dispersibility, wherein X represents an amount of an acid in a coagulation bath, and the amount is between 0 and 100%.

In one embodiment, the intrinsic dispersibility of the ncrys-PEDOT_{X} enables its homogeneous incorporation at desired loading within diverse aqueous biomaterial solutions without additives or surfactants.

In one embodiment, the desired loadings is between 0-100%, preferably, varying within 0-5%, 5-10%, 10-20%, . . . up to 99%.

In one embodiment, the ncrys-PEDOT_{X} is directly incorporable within a hydrogel, a scaffold, a film, a hydrophobic polymer, an elastomer, a thermoplastic, thermoset and/or an aqueous resin formation without sonication or external surfactants.

In one embodiment, the ncrys-PEDOT_{X} is biocompatible, and hemocompatible.

In one embodiment, the ncrys-PEDOT_{X} is usable in scalable, conductive, biocompatible, and modular platforms for bioelectronic applications.

In one embodiment, the ncrys-PEDOT_{X} is synthesized with an acid-based nonsolvent induced phase separation (NIPS) method by coagulating a PEDOT:PSS solution into stable aggregates of concentrated PEDOT with tuneable PSS surfactant.

In one embodiment, the PEDOT:PSS solution comprises PEDOT:PSS added in a coagulation bath including acid in isopropanol (IPA). In one embodiment, the acid is a sulfuric acid. The sulfuric acid is adapted to remove the PEDOT from the PSS, while the IPA is used to act as a nonacidic solvent to wash away the removed PSS. It should be noted that many other acids and many other organic solvents can also be utilized to practice the invention.

In one embodiment, increasing the concentrations of the acid within the coagulation bath correlates with increasing a PEDOT/PSS ratio.

In one embodiment, the degree to which PSS is removed and PEDOT crystallizes within the ncrys-PEDOT_{X} particles is directly related to the concentration of the sulfuric acid within the coagulation bath.

In one embodiment, by tuning a volume ratio of sulfuric acid within the coagulation bath, the PEDOT/PSS ratio in the ncrys-PEDOT_{X} is optimized to afford the ncrys-PEDOT_{X} with high conductivity (σ_{ncrys-PEDOT20}=410 S cm⁻¹) rivaling all the existing conjugated polymer particles.

In one embodiment, the ncrys-PEDOT_{X} has conductivities in a range of about 1-100 S cm⁻¹, about 100-400 S cm⁻¹, and/or about 400-800 S cm⁻¹.

In one embodiment, the ncrys-PEDOT₅ and ncrys-PEDOT₂₀ have conductivities of about 87 and 410 S cm⁻¹. The exemplary embodiment shows X=5% and =20% as two representative examples. It should be noted that many other concentrations of the acid, for example, X=1, 2, 3, 4%, . . . , or all the way to 100% acid concentration, can also be used to practice the invention. All these concentrations would change the final properties of the ncrys-PEDOT_{X} particles. In one embodiment, crystallization of the ncrys-PEDOT_{X} particles enhances with increasing the concentration of the sulfuric acid within the coagulation bath.

In one embodiment, a crystallite size of the ncrys-PEDOT_{X} particles increases from about 1.26 to 1.58 nm upon treatment with more concentrated sulfuric acid.

In one embodiment, the ncrys-PEDOT_{X} particles have not interfered with chemistries of crosslink hydrogels including hydrogen bonding, ionic bonding, Schiff-base chemistry, and radical photopolymerization.

In one embodiment, by directly adding the ncrys-PEDOT_{X} to a hydrogel formulation, a highly conductive composite is achieved with a percolation threshold between 0-5, 5-10, 10-15, or 15-20 wt % loading of the ncrys-PEDOT_{X}.

In one embodiment, the PEDOT:PSS is formed with one or more functional monomers to make the ncrys-PEDOT_{X} particles to have greater functionality.

In another aspect, the invention relates to a method of synthesizing a conductive nanomaterial. The methods comprises dropwisely adding an aqueous solution of PEDOT:PSS into a coagulation bath including sulfuric acid in isopropanol (IPA) to form a mixture thereof; collecting particles from the mixture; and subsequently comminuting the collected particles into a fine powder to form the conductive nanomaterial comprising acid-crystalized ncrys-PEDOT_{X} with intrinsic dispersibility while maintaining high conductivity, wherein X represents an amount of the acid in the coagulation bath, and the amount is between 0 and 100%.

In one embodiment, the IPA is a nonsolvent that dehydrates the colloidal dispersion, enabling the NIPS.

In one embodiment, the sulfuric acid is to stabilize segregation of insulating PSS from conductive PEDOT.

In one embodiment, conductivity enhancement from acid crystallization involves removal of PSS and enhanced crystallinity.

In one embodiment, increasing a concentration of the sulfuric acid within the coagulation bath correlates with increasing a PEDOT/PSS ratio in the ncrys-PEDOT_{X}.

In one embodiment, the degree to which PSS is removed and PEDOT crystallizes within the ncrys-PEDOT_{X} particles is directly related to the concentration of the sulfuric acid within the coagulation bath.

In one embodiment, by tuning a volume ratio of sulfuric acid within the coagulation bath, the PEDOT/PSS ratio in the ncrys-PEDOT_{X} is optimized to afford the ncrys-PEDOT_{X} with high conductivity (σ_{ncrys-PEDOT20}=410 S cm⁻¹) rivaling all the existing conjugated polymer particles.

In one embodiment, the ncrys-PEDOT₅ and ncrys-PEDOT₂₀ particles represent conditions with low and high concentrations of sulfuric acid in the coagulation bath, respectively.

In one embodiment, the ncrys-PEDOT₅ and ncrys-PEDOT₂₀ have conductivities of about 87 and 410 S cm⁻¹.

In one embodiment, crystallization of the ncrys-PEDOT_{X} particles enhances with increasing the concentration of the sulfuric acid within the coagulation bath.

In one embodiment, a crystallite size of the ncrys-PEDOT_{X} particles increases from about 1.26 to 1.58 nm upon treatment with more concentrated sulfuric acid.

In a further aspect, the invention relates to a composite comprising an aqueous biomaterial solution; and acid-crystalized ncrys-PEDOT_{X} loaded into the aqueous biomaterial solution, wherein the ncrys-PEDOT_{X} acts as a filler with the highest conductivity of the particles while maintaining dispersibility, wherein X represents an amount of an acid in a coagulation bath, and the amount is between 0 and 100%.

In one embodiment, the aqueous biomaterial solution is devoid of additives or surfactants.

In one embodiment, the ncrys-PEDOT_{X} is loaded into the aqueous biomaterial solution by vortexing, stirring, shaking, centrifugation, milling, and/or the likes.

In one embodiment, the aqueous biomaterial solution comprises a hydrogel synthesized from aqueous solutions of hydrophilic natural biopolymers including alginate, gelatin, collagen, and/or chitosan, or synthetic polymer formulations including pHEMA, PEO, and/or PEGDA.

In one embodiment, the composite has a significant increase in conductivity with a percolation threshold between 15 and 20 wt % loading of the ncrys-PEDOT_{X}, with the highest loading displaying a remarkable conductivity of 1.1 S cm⁻¹.

In one embodiment, the composite has about 8.2 S cm⁻¹ with 5% particle loading, which is notably >400 fold higher than an EDOT postpolymerization in PEDGA alone (0.02 S cm⁻¹).

In one embodiment, the ncrys-PEDOT_{X} has no interfered with chemistries of the aqueous biomaterial solution.

In one embodiment, the ncrys-PEDOT_{X} has no deleterious impact on the stability of the aqueous biomaterial solution.

In one embodiment, the composite comprises an aqueous photoprintable conductive resin is formulated through the addition of the ncrys-PEDOT_{X} into a mixture of poly(ethylene glycol)diacrylate (PEGDA) and lithium phenyl (2,4,6-trimethylbenzoyl) phosphinate (LAP) photoinitiator in water.

In one embodiment, the aqueous PEGDA resin is utilized for 3D printing of a soft matter with complex form factors with high fidelity complex biomedical structures for biomedical applications.

In one embodiment, the composite has high cell viability (>95%) at all loadings up to about 15% of ncrys-PEDOT_{X}.

In one embodiment, the composite is biocompatible and/or hemocompatibible.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows schematically dispersible nanoparticles of crys-PEDOT_{X} synthesized from PEDOT:PSS according to embodiments of the invention. PEDOT:PSS solution (panel a) can be converted into dispersible acid-crystallized nanoparticles (ncrys-PEDOT_{X}) by (panel b) nonsolvent induced phase separation (NIPS). Panel c: SEM image of particles coagulated in an acid bath with 20% sulfuric acid (ncrys-PEDOT₂₀) with a corresponding histogram displaying particle dimension statistics. Panel d: S 2p high resolution XPS spectra including fits for PEDOT (orange) and PSS (gray). PEDOT/PSS ratio was calculated from an area comparison of the S 2p fits. Panel e: X-ray diffraction (XRD) patterns of ncrys-PEDOT₅ and ncrys-PEDOT₂₀, focused on the (010) reflections corresponding to π-π stacking between adjacent PEDOT chains. Panel f: Current-voltage curves of pelletized samples of ncrys-PEDOT_{X} particles are characterized by ohmic behavior; the inset shows a representative disc-shaped ncrys-PEDOT₂₀ pellet. Panel g: Conductivity comparison of ncrys-PEDOT_{X} with other reported conjugated polymer nanoparticles from Table 1.

FIG. 2 shows schematically dispersibility of ncrys-PEDOT₂₀ within conductive hydrogels according to embodiments of the invention. Panel a: Sedimentation kinetics of 10 mg mL⁻¹ ncrys-PEDOT₂₀ in DI H₂O, 2% aqueous sodium alginate, and 2% aqueous chitosan solutions were monitored by the change in absorbance at the meniscus of the particle-incorporated solutions. Panel b: Conductive hydrogels prepared by simply adding the ncrys-PEDOT₂₀ to aqueous biomaterial dispersions, stirring the mixture, and curing the hydrogel via thermal, covalent, ionic, or photoinitiated crosslinking methodologies. Images display the particle loaded dispersions before (left) and after (right) crosslinking. For the particle loaded alginate dispersion, crosslinking was demonstrated by the “alginate-worm experiment” within a solution of 1_{M} CaCl₂.

FIG. 3 shows schematically conductivity characterization of conductive hydrogels with incorporated ncrys-PEDOT₂₀ according to embodiments of the invention. Panel a: Conductivity of photoinitiated hydrogels with square form factors via four-point probe analysis. Conductivity was measured on hydrogels with increasing loading of ncrys-PEDOT₂₀ with (dark blue outline) and without subsequent in situ polymerization of EDOT. The cartoons represent percolation being achieved through either (left) connection of particles via PEDOT from in situ postpolymerization or (right) particle contact through elevated concentration. The dotted lines represent conductivity at 0 wt % particle loading. Panel b: Conductivity comparison of ncrys-PEDOT₂₀ loaded PEGDA hydrogel from panel a with other reported conductive hydrogels from Table 2. Each example is categorized by the order of magnitude of the reported conductivity (x-axis), and by the base composition of the hydrogel.

FIG. 4 shows schematically μCLIP 3D printing of ncrys-PEDOT_{X} according to embodiments of the invention. Panel a: ncrys-PEDOT₂₀ can be dispersed within aqueous resins for 3D printing conducting hydrogels. Particles were added to a photocurable resin which consisted of poly(ethylene glycol)diacrylate (PEGDA), lithium phenyl (2,4,6-trimethylbenzoyl) phosphinate (LAP), and water. Panel b: Diagram of the microcontinuous liquid interface production (μCLIP) stereolithography process to generate hydrogels. (Right) Image of the homemade μCLIP printer used to print all structures. Panel c: Sedimentation kinetics of the PEGDA resin with 10 wt % loading of ncrys-PEDOT₂₀ (100 mg mL⁻¹) demonstrating stable dispersions within the timeframe of a print. (Inset) Image of the particle-loaded resin after sitting for 1 h. Panel d: Cell viability of L929 cells treated with either media alone or extracts of 3D printed ncrys-PEDOT20 incorporated hydrogels after incubation for 48 h. Panel e: Quantitative measurement of cell viability using live/dead assay after incubation for 48 h. Panels f-i: Fluorescent images of cells taken after live/dead assay, where calcein AM (green) represents live cells and ethidium homodimer (red) represents dead cells in contact with each ncrys-PEDOT₂₀ incorporated hydrogel. Scale bars for the images are 100 μm. (inset) Image of particle loaded hydrogel with the square form factor used for both four-point probe and biocompatibility analysis. Panel j: Renderings of a complex tubular scaffold with a form factor consistent for an esophageal stent with 133.33 μm diameter struts. Photography (left) and SEM imaging (right) of PEGDA stents with k) 5 wt % and 1) 10 wt % loading of ncrys-PEDOT₂₀. Scale bars for the photographs and SEM images are 2 mm.

FIG. 5 shows schematically the strong absorption features in the UV attributed to phenyl moieties within the PSS, which are reduced significantly in particles processed in coagulation baths with increasing concentrations of sulfuric acid. Particles coagulated in baths including 50% sulfuric acid (ncrys-PEDOT₅₀) lacked the dispersibility to be analyzed by UV/Vis at 1 mg mL⁻¹ concentrations, suggesting that too much crystallization and removal of PSS may lead to limited aqueous stability.

FIG. 6 shows (panel a) sedimentation kinetics of 10 mg mL⁻¹ ncrys-PEDOT₅ and ncrys-PEDOT₂₀ solutions in DI H₂O and 2% aqueous sodium alginate, and (panel b) images of dispersion sedimentation at different time points.

FIG. 7 shows (panel a) sedimentation kinetics of 10 mg mL⁻¹ ncrys-PEDOT₂₀ solutions in 0.2% aqueous collagen, and (panel b) images of dispersion sedimentation at different time points.

FIG. 8 shows (panel a) swelling of alginate hydrogels with and without 1 wt % of ncrys-PEDOT₂₀, as calculated by the change in mass from water uptake over a 48-hour period, and (panel b) conductivity of the alginate gels as measured by four-point probe conductivity in the swollen state.

FIG. 9 shows (panel a) compressive stress-strain behavior of alginate hydrogels with and without 1 wt % ncrys-PEDOT₂₀, and (panel b) Young's modulus of the alginate hydrogels. (c) Relative increase in the Young's modulus (i.e., modulus reinforcement) for both alginate and chitosan hydrogels when loaded with 1 wt % of ncrys-PEDOT₂₀. The average modulus reinforcement for reduced graphene oxide (rGO) and carbon nanotubes (CNTs) are annotated for comparison.

FIG. 10 shows (panel a) electrochemical analysis of the swollen alginate hydrogel with 1 wt % loading of ncrys-PEDOT₂₀ under an applied stress via EIS. Analysis was performed at varying distances between the two electrodes—these distances were chosen to maintain hydrogel contact with the electrodes while preventing physical damage to the hydrogel structure. Panel b: After fitting data to equivalent circuits, the conductivity was calculated for the gel at each electrode distance (i.e., hydrogel thickness)—the measured conductivity was enhanced under compression, which we hypothesize is due to enhanced charge percolation from a decreased interparticle distance. The inset shows the EIS setup made from a Swagelok cell to control hydrogel thickness.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Conductive hydrogels are promising materials with mixed ionic-electronic conduction to interface living tissue (ionic signal transmission) with medical devices (electronic signal transmission). The hydrogel form factor also uniquely bridges the wet/soft biological environment with the dry/hard environment of electronics. The synthesis of hydrogels for bioelectronics requires scalable, biocompatible fillers with high electronic conductivity and compatibility with common aqueous hydrogel formulations/resins. Despite significant advances in the processing of carbon nanomaterials, fillers that satisfy all these requirements are lacking.

In view of the foregoing, one aspect of the invention relates to intrinsically dispersible acid-crystalized PEDOT:PSS nanoparticles (ncrys-PEDOT_{X}) which are processed through a facile and scalable nonsolvent induced phase separation method from commercial PEDOT:PSS without complex instrumentation. The particles feature conductivities of up to 410 S cm⁻¹, and when compared to other common conductive fillers, display remarkable dispersibility, enabling homogeneous incorporation at relatively high loadings within diverse aqueous biomaterial solutions without additives or surfactants. The aqueous dispersibility of the ncrys-PEDOT_{X} particles also allows simple incorporation into resins designed for microstereolithography without sonication or surfactant optimization; complex biomedical structures with fine features (<150 μm) are printed with up to 10% particle loading. The ncrys-PEDOT_{X} particles overcome the challenges of traditional conductive fillers, providing a scalable, biocompatible, plug-and-play platform for soft organic bioelectronic materials.

Without intent to limit the scope of the invention, exemplary embodiments of the present invention are described below.

One aspect of the invention relates to a conductive nanomaterial comprising acid-crystalized ncrys-PEDOT_{X} with intrinsic dispersibility, wherein X represents an amount of an acid in a coagulation bath, and the amount is between 0 and 100%.

In one embodiment, the intrinsic dispersibility of the ncrys-PEDOT_{X} enables its homogeneous incorporation at desired loading within diverse aqueous biomaterial solutions without additives or surfactants.

In one embodiment, the desired loadings is between 0-100%, preferably, varying within 0-5%, 5-10%, 10-20%, . . . up to 99%.

In one embodiment, the ncrys-PEDOT_{X} is directly incorporable within a hydrogel, a scaffold, a film, a hydrophobic polymer, an elastomer, a thermoplastic, thermoset and/or an aqueous resin formation without sonication or external surfactants.

In one embodiment, the ncrys-PEDOT_{X} is biocompatible, and hemocompatible.

In one embodiment, the ncrys-PEDOT_{X} is usable in scalable, conductive, biocompatible, and modular platforms for bioelectronic applications.

In one embodiment, the ncrys-PEDOT_{X} is synthesized with an acid-based nonsolvent induced phase separation (NIPS) method by coagulating a PEDOT:PSS solution into stable aggregates of concentrated PEDOT with tuneable PSS surfactant.

In one embodiment, the PEDOT:PSS solution comprises PEDOT:PSS added in a coagulation bath including acid in isopropanol (IPA). In one embodiment, the acid is a sulfuric acid. The sulfuric acid is adapted to remove the PEDOT from the PSS, while the IPA is used to act as a nonacidic solvent to wash away the removed PSS. It should be noted that many other acids and many other organic solvents can also be utilized to practice the invention.

In one embodiment, increasing the concentrations of the acid within the coagulation bath correlates with increasing a PEDOT/PSS ratio.

In one embodiment, the degree to which PSS is removed and PEDOT crystallizes within the ncrys-PEDOT_{X} particles is directly related to the concentration of the sulfuric acid within the coagulation bath.

In one embodiment, by tuning a volume ratio of sulfuric acid within the coagulation bath, the PEDOT/PSS ratio in the ncrys-PEDOT_{X} is optimized to afford the ncrys-PEDOT_{X} with high conductivity (σ_{ncrys-PEDOT20}=410 S cm⁻¹) rivaling all the existing conjugated polymer particles.

In one embodiment, the ncrys-PEDOT_{X} has conductivities in a range of about 1-100 S cm⁻¹, about 100-400 S cm⁻¹, and/or about 400-800 S cm¹.

In one embodiment, the ncrys-PEDOT₅ and ncrys-PEDOT₂₀ have conductivities of about 87 and 410 S cm⁻¹. The exemplary embodiment shows X=5% and =20% as two representative examples. It should be noted that many other concentrations of the acid, for example, X=1, 2, 3, 4%, . . . , or all the way to 100% acid concentration, can also be used to practice the invention. All these concentrations would change the final properties of the ncrys-PEDOT_{X} particles. In one embodiment, crystallization of the ncrys-PEDOT_{X} particles enhances with increasing the concentration of the sulfuric acid within the coagulation bath.

In one embodiment, a crystallite size of the ncrys-PEDOT_{X} particles increases from about 1.26 to 1.58 nm upon treatment with more concentrated sulfuric acid.

In one embodiment, the ncrys-PEDOT_{X} particles have not interfered with chemistries of crosslink hydrogels including hydrogen bonding, ionic bonding, Schiff-base chemistry, and radical photopolymerization.

In one embodiment, by directly adding the ncrys-PEDOT_{X} to a hydrogel formulation, a highly conductive composite is achieved with a percolation threshold between 0-5, 5-10, 10-15, or 15-20 wt % loading of the ncrys-PEDOT_{X}.

In one embodiment, the PEDOT:PSS is formed with one or more functional monomers to make the ncrys-PEDOT_{X} particles to have greater functionality.

Another aspect of the invention relates to a method of synthesizing a conductive nanomaterial. The methods comprises dropwisely adding an aqueous solution of PEDOT:PSS into a coagulation bath including sulfuric acid in isopropanol (IPA) to form a mixture thereof; collecting particles from the mixture; and subsequently comminuting the collected particles into a fine powder to form the conductive nanomaterial comprising acid-crystalized ncrys-PEDOT_{X} with intrinsic dispersibility while maintaining high conductivity, wherein X represents an amount of the acid in the coagulation bath, and the amount is between 0 and 100%.

In one embodiment, the IPA is a nonsolvent that dehydrates the colloidal dispersion, enabling the NIPS.

In one embodiment, the sulfuric acid is to stabilize segregation of insulating PSS from conductive PEDOT.

In one embodiment, conductivity enhancement from acid crystallization involves removal of PSS and enhanced crystallinity.

In one embodiment, increasing a concentration of the sulfuric acid within the coagulation bath correlates with increasing a PEDOT/PSS ratio in the ncrys-PEDOT_{X}.

In one embodiment, the degree to which PSS is removed and PEDOT crystallizes within the ncrys-PEDOT_{X} particles is directly related to the concentration of the sulfuric acid within the coagulation bath.

In one embodiment, by tuning a volume ratio of sulfuric acid within the coagulation bath, the PEDOT/PSS ratio in the ncrys-PEDOT_{X} is optimized to afford the ncrys-PEDOT_{X} with high conductivity (σ_{ncrys-PEDOT20}=410 S cm⁻¹) rivaling all the existing conjugated polymer particles.

In one embodiment, the ncrys-PEDOT₅ and ncrys-PEDOT₂₀ particles represent conditions with low and high concentrations of sulfuric acid in the coagulation bath, respectively.

In one embodiment, the ncrys-PEDOT₅ and ncrys-PEDOT₂₀ have conductivities of about 87 and 410 S cm⁻¹.

In one embodiment, crystallization of the ncrys-PEDOT_{X} particles enhances with increasing the concentration of the sulfuric acid within the coagulation bath.

In one embodiment, a crystallite size of the ncrys-PEDOT_{X} particles increases from about 1.26 to 1.58 nm upon treatment with more concentrated sulfuric acid.

A further aspect of the invention relates to a composite comprising an aqueous biomaterial solution; and acid-crystalized ncrys-PEDOT_{X} loaded into the aqueous biomaterial solution, wherein the ncrys-PEDOT_{X} acts as a filler with the highest conductivity of the particles while maintaining dispersibility, wherein X represents an amount of an acid in a coagulation bath, and the amount is between 0 and 100%.

In one embodiment, the aqueous biomaterial solution is devoid of additives or surfactants.

In one embodiment, the ncrys-PEDOT_{X} is loaded into the aqueous biomaterial solution by vortexing, stirring, shaking, centrifugation, milling, and/or the likes.

In one embodiment, the aqueous biomaterial solution comprises a hydrogel synthesized from aqueous solutions of hydrophilic natural biopolymers including alginate, gelatin, collagen, and/or chitosan, or synthetic polymer formulations including pHEMA, PEO, and/or PEGDA.

In one embodiment, the composite has a significant increase in conductivity with a percolation threshold between 15 and 20 wt % loading of the ncrys-PEDOT_{X}, with the highest loading displaying a remarkable conductivity of 1.1 S cm⁻¹.

In one embodiment, the composite has about 8.2 S cm⁻¹ with 5% particle loading, which is notably >400 fold higher than an EDOT postpolymerization in PEDGA alone (0.02 S cm⁻¹).

In one embodiment, the ncrys-PEDOT_{X} has no interfered with chemistries of the aqueous biomaterial solution.

In one embodiment, the ncrys-PEDOT_{X} has no deleterious impact on the stability of the aqueous biomaterial solution.

In one embodiment, the composite comprises an aqueous photoprintable conductive resin is formulated through the addition of the ncrys-PEDOT_{X} into a mixture of poly(ethylene glycol)diacrylate (PEGDA) and lithium phenyl (2,4,6-trimethylbenzoyl) phosphinate (LAP) photoinitiator in water.

In one embodiment, the aqueous PEGDA resin is utilized for 3D printing of a soft matter with complex form factors with high fidelity complex biomedical structures for biomedical applications.

In one embodiment, the composite has high cell viability (>95%) at all loadings up to about 15% of ncrys-PEDOT_{X}.

In one embodiment, the composite is biocompatible and/or hemocompatibible.

These and other aspects of the present invention are further described below. Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example

### Conducting Polymer Nanoparticles with Intrinsic Aqueous Dispersibility for Conductive Hydrogels

In this exemplary study, we report intrinsically dispersible acid-crystalized PEDOT:PSS nanoparticles (ncrys-PEDOT_{X}) to enable 3D charge percolation within hydrogels for organic bioelectronic applications. Using an acid-based nonsolvent induced phase separation (NIPS) method, commercial PEDOT:PSS inks were coagulated into stable aggregates of concentrated PEDOT with tuneable PSS surfactant to enable dispersion within prototypical hydrogels. Compared to other common conductive fillers ncrys-PEDOT_{X} displays enhanced dispersibility, enabling homogeneous incorporation at relatively high loadings within diverse aqueous biomaterial solutions without additives or surfactants. To demonstrate the plug and play nature of the particles ncrys-PEDOT₂₀ was directly incorporated within both natural biomaterial derived hydrogels and an aqueous resin designed for projection microstereolithography without sonication or surfactant optimization. With only a single step for conductive filler incorporation, hydrogels were endowed with significant conductivity (>1 S cm⁻¹) rivaling other reported systems in performance and simplicity. Furthermore, high fidelity complex biomedical structures were printed with up to 10% loading of conductive particles with fine features (<150 μm), biocompatibility, and hemocompatibility. The ncrys-PEDOT_{X} particles overcome the challenges of traditional conductive fillers, offering a scalable, conductive, biocompatible, and modular platform for soft organic bioelectronic materials.

### Materials and Methods

Materials: PEDOT:PSS aqueous solution (Clevios PH1000) was purchased from Heraeus Electronic Materials. PEDOT:PSS dry redispersible pellets, sulfuric acid (ACS reagent, 95.0-98.0%), gelatin from porcine skin (gel strength 300, Type A), chitosan (medium molecular weight), agarose (Type I, low EEO), sodium alginate, and poly(ethylene glycol) diacrylate (PEGDA, Mₙ=575 g mol⁻¹) were purchased from Sigma-Aldrich and used as received. Rat tail collagen (Type I, 2.05 mg mL⁻¹ in 0.6% acetic acid) was purchased from First Link (UK) Ltd. Glutaraldehyde (≈50% in water, ≈5.6 mol L⁻¹) and lithium phenyl(2,4,6-trimethylbenzoyl)phosphinate (LAP) were purchased from TCI.

Preparation of ncrys-PEDOT_{X} Nanoparticles: Acid crystallized PEDOT:PSS nanoparticles (ncrys-PEDOT_{X}) were synthesized by dropwise addition of PEDOT:PSS aqueous solution into a coagulation bath including sulfuric acid in isopropanol under stirring for 12 h. Subsequently the particles were filtered, washed with isopropanol, deionized water, and acetone. The particles were then dried in vacuo and milled (Freezer/Mill SPEX Cryogenic Grinder) for three cycles where one cycle included 5 min of precool time, 15 min of grinding, and 2 min of rest. The speed was maintained at a rate of 15 counts per second (cps). Crystallization of 100 mL of ink within a 20% acid bath yielded 802 mg of ncrys-PEDOT₂₀.

Size Analysis: To probe the size distribution and morphology of the PEDOT nanoparticles, scanning electron microscopy was conducted using a Hitachi S-4800 SEM operated at 5 kV. Nanoparticles were dispersed in methanol and were drop cast and air-dried on carbon-coated tapes prior to imaging. The high conductivity of the particles precluded the need for a secondary conductive coating. The resulting images were analyzed using ImageJ, data were collated from over 200 individual particles.

X-Ray Photoelectron Spectroscopy: Measurements were performed with a NEXSA G2 XPS instrument, using a 15 keV Al-Kα X-ray source. Survey and high-resolution spectra of C 1s and S 2p core levels were recorded. All spectra were adjusted to the C is level of 284.8 eV. Data analysis was performed using the Thermo Fisher Avantage software.

X-Ray Diffraction: PXRD data were collected at room temperature on a STOE-STADI-P powder diffractometer equipped with an asymmetric curved Germanium monochromator (Cu Kα1 radiation, λ=1.54056 Å) and 1D silicon strip detector (MYTHEN2 1K from DECTRIS). The line focused Cu X-ray tube was operated at 40 kV and 40 mA. Powder was packed in an 8 mm metallic mask and sandwiched between two polyimide layers of tape. Intensity data from 1.5° to 70° two theta were collected over a period of 120 min. Instrument was calibrated against a NIST Silicon standard (640d) prior the measurement.

Conductivity Measurements: For particle conductivity measurements 50 mg of ncrys-PEDOT_{X} or commercial lyophilized PEDOT:PSS was pressed into a pellet with an 8 mm diameter by pressing under a hydrostatic pressure of 3500 kg cm⁻² for 2 min. Four-point conductivity measurements were performed using a Lucas Labs Pro4 four-point probe head with a Keithley 9 2400 source meter. Samples were measured in triplicate and the average pellet or gel thickness was determined via caliper.

Sedimentation Analysis: Dispersions were prepared using 10 mg mL⁻¹ concentrations of either ncrys-PEDOT₅ or ncrys-PEDOT₂₀ in either DI H₂O and 2 wt % sodium alginate in DI H₂O. Dispersions (10 mg mL⁻¹) of ncrys-PEDOT₂₀ were also prepared in 2 wt % chitosan in 1% acetic acid and 0.2 wt % collagen in 0.6% acetic acid. At predetermined time intervals, aliquots of the supernatants were carefully collected from just below the surface of the meniscus and analyzed via UV-Vis at 633 nm. The ratio of the absorbance at a given time point compared to that of the initial time point was used to measure the stability of the suspensions.

Hydrogel Preparation: Gelatin: 400 mg of gelatin was added to 20 mL of DI H₂O and stirred at 45° C. for 1 h. A solution of ncrys-PEDOT₂₀ (10 mg mL⁻¹) was prepared using the gelatin stock solution and stirred at 45° C. for 1 h. For the vial tilt experiment, 4 mL of particle-loaded gelatin dispersion was slowly cooled to room temperature. For the reinforcement experiment, 6 mL of the particle-loaded gelatin dispersion was poured into a polystyrene dish and slowly cooled to room temperature. Discs were punched using an 8 mm biopsy punch and washed with DI H₂O. Alginate: 400 mg of sodium alginate was added to 20 mL of DI H₂O and stirred at room temperature overnight. A solution of ncrys-PEDOT₂₀ (10 mg mL⁻¹) was prepared using the alginate stock solution and stirred at room temperature for 1 h. For the alginate worm experiment, particle-loaded alginate dispersion was injected into an aqueous solution of 1 M CaCl₂. For the reinforcement experiment 6 mL of the particle-loaded alginate dispersion was poured into a polystyrene dish lined with a filter paper soaked with 0.05 M CaCl₂ and incubated for 24 h at room temperature according to previously reported procedures. Discs were punched using an 8 mm biopsy punch and washed with DI H₂O. Chitosan: 2 g of chitosan was added to 100 mL of aqueous acetic acid (1% v/v) and stirred at room temperature until homogeneous. A solution of ncrys-PEDOT₂₀ (10 mg mL⁻¹) was prepared using the chitosan stock solution and stirred at room temperature for 1 h. For the vial tilt experiment 1 mL of aqueous glutaraldehyde (50%) was added to 4 mL of particle loaded chitosan dispersion at room temperature. For the reinforcement experiment 6 mL of the particle loaded chitosan dispersion was poured into a polystyrene dish lined with a filter paper soaked in 25% aqueous glutaraldehyde and incubated for 24 h at room temperature. Discs were punched using an 8 mm biopsy punch and washed with DI H₂O.

Mechanical Characterization: The mechanical properties of the hydrogels were investigated in the compression mode using a DMA (850, TA Instruments) with a 0.05 N preload force, at 25° C. under immersion in DI H₂O. The elastic modulus was determined from slope of the stress-strain curve within the elastic region. The elastic modulus, compressive strength, and compressive strain were calculated from five hydrogels discs.

Printing of ncrys-PEDOT₂₀ Incorporated Resins: A home-made μCLIP printer was used to print all structures utilized throughout the study. Conditions used for printer setup and operation followed previously reported procedures. Focal plane pixel resolution was set at 3.98×3.98 μm, and 5 μm layer thickness was used. The system is similar to those previously described, with updates to the light projector and light path. A hydrogel resin including PEGDA (39.5 wt %), LAP (0.5 wt %), and water (60 wt %) was used with varying loading of ncrys-PEDOT₂₀. Particles were loaded into the resin at 1, 5, or 10 wt %, initially vortexed for 2 min, and vortexed for 1 min immediately both before use and between each print. Using the μCLIP printer, esophageal stents and square form factors were printed. Esophageal stents were designed to be 10.93 mm long with supports and have struts 133 μm in diameter. Squares were 8 mm in length and height and were 1 mm in thickness, with 1 mm tall supports on one side to adhere prints to the platform. Inks with 1 wt % ncrys-PEDOT₂₀ were printed with 0.0008 mJ mm⁻² power, 5 wt % loading with 0.2188 mJ mm⁻² power, and 10 wt % loading with 0.3063 mJ mm⁻² power. In situ polymerization of particle loaded prints with EDOT followed previously reported procedures.

Biocompatibility Analysis: In vitro cytotoxicity tests were performed on particle-loaded PEGDA hydrogels based on the ISO 10993-5 protocol. 3D printed hydrogels with square (8 mm×8 mm×1 mm) form factors were assessed with either 1, 5, 10, or 15 wt % ncrys-PEDOT₂₀. Indirect Extract Tests: Before performing cytotoxicity experiments, each hydrogel was washed with ethanol, DPBS, and Dulbecco's Modified Eagle Medium/Nutrient Mixture F-12 (DMEM/F-12) media (Gibco; Cat no. 11 320 033). DMEM/1F-12 media supplemented with both 10% fetal bovine serum (FBS) and 1% antibiotic-antimycotic (2 mL) was added directly onto 0.15 g of hydrogel and incubated at 37° C. with 5% CO₂ in a humidified incubator for 24 h to create extracts. Separately, a suspension of L929 cells was prepared at a concentration of 5×10⁴ cell mL⁻¹ and 100 μL of the suspension was dispensed into each well of a 96-well plate. The plate was incubated at 37° C. with 5% CO₂ in a humidified incubator. After 24 h, culture media was removed from the wells and replaced by 100 μL of extract media from the hydrogels. Following 48 h, the cell viability was assessed via alamarBlue assay (Invitrogen; Cat no. A50101). Samples were repeated in quintuplicate. Direct Tests: L929 cells were prepared at a concentration of 5×10⁴ cell mL⁻¹ and 200 μL of this suspension was added directly on the particle-incorporated hydrogels within a 24-well polystyrene tissue culture plate. The plate was incubated at 37° C. with 5% CO₂ in a humidified incubator for 4 h for cell attachment, and 300 μL of DMEM/F-12 media supplemented with both 10% fetal bovine serum (FBS) and 1% antibiotic-antimycotic was carefully added into each well. Following 48 h, a live/dead solution was prepared from the LIVE/DEAD Viability/Cytotoxicity Kit (Invitrogen; Cat no. L3224) with 0.5 μL of calcein AM and 2.0 μL of ethidium homodimer-1 (EthD-1) in 1 mL of DPBS. Each hydrogel was stained with live/dead solution (200 μL), incubated for 30 min, and imaged with a Nikon confocal microscope. The cell viability percentage was calculated using Fiji (ImageJ).

Hemocompatibility Analysis: 12 mL of the blood samples were collected from three female rabbits and pooled together in anticoagulant vials. The hemocompatibility of ncrys-PEDOT₂₀ was assessed using a 50 mg mL⁻¹ stock solution in DI water. The hemolysis assay protocol was adapted from prior literature procedures. Briefly, 300 μL of particle solution was added to 4 mL of 0.9% saline solution and equilibrated for 30 min at 37° C. 200 μL of diluted blood (4 mL of blood diluted in 5 mL of 0.9% saline solution) was added to the sample. A negative control was prepared by adding 200 μL of diluted blood to 4 mL of 0.9% saline solution (0% hemolysis) and a positive control was prepared by adding 200 μL of diluted blood to 4 mL of DI water (100% hemolysis). The sample was then incubated at 37° C. for 1 h, centrifuged at 1000 rpm for 5 min, and the absorbance of the supernatant was measured at 545 nm. The hemolysis experiment was performed in triplicate.

### Results and Discussion

Dispersible and Tunable ncrys-PEDOT_{X}—Preparation and Characterization: The synthesis of PEDOT nanoparticles is typically achieved through bottom-up, template-, or emulsion-based methods which suffer from limited tunability, low yields, and particles that lack aqueous processability, the latter of which is a critical requirement when working with biomaterial dispersions. While reactors have been reported to afford submicron PEDOT particles via aerosol vapor polymerization and electrochemistry, such methods require sophisticated instrumentation and, as the afforded particles lack a polyanionic surfactant such as PSS, have both limited aqueous dispersibility and require external dopants (Table 1). Alternatively, commercial PEDOT:PSS can be concentrated via lyophilization and redispersed within a biomaterial; while this top-down approach is scalable and accessible due to the commercial availability of PEDOT:PSS, the afforded composites demonstrate limited conductivity as well as leeching over time due to the excess insulating PSS dispersant. Recently an acid-based nonsolvent induced phase separation (NIPS) approach was applied to coagulate PEDOT:PSS into stable, microparticles to both partially remove insulating PSS and enhance the crystallinity of the PEDOT assemblies, however this method afforded porous systems with size features too large for dispersion within conductive hydrogels. Acid treatment has also been recently implemented to improve and investigate mixed conduction within thin film devices.

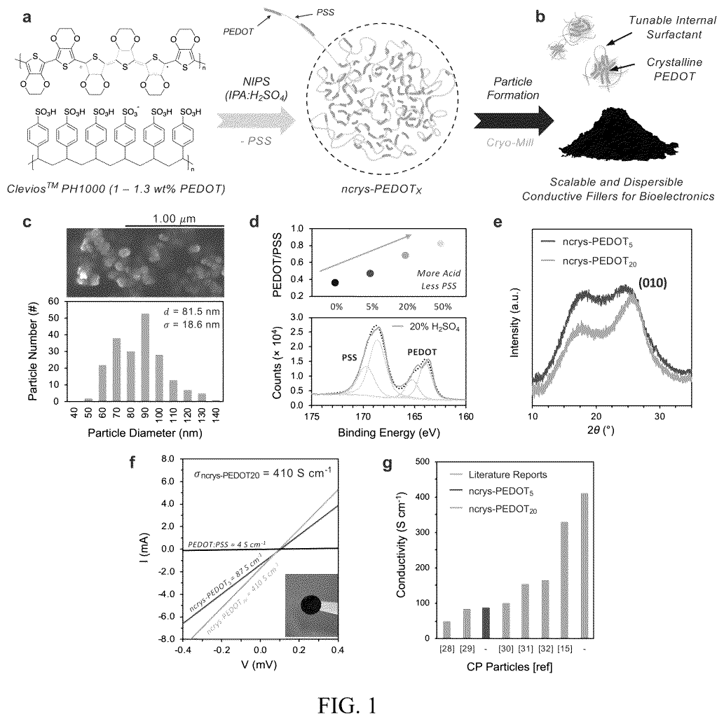

In this exemplary example, we leverage a top-down NIPS-based processing approach to commercial PEDOT:PSS (Heraeus Electronic Materials, 1.1-1.3% solid content, Clevios PH1000) to prepare tuneable PEDOT:PSS nanoparticles with intrinsic dispersibility for conductive hydrogel applications, as shown in panel a of FIG. 1. The novel method is designed to be simple for adoption by nonchemists and affords particles on the multigram scale within an academic laboratory setting, as shown in panel b of FIG. 1.

TABLE 1
Literature Comparison of Sub-Micron PEDOT Particles

| | | | Water | | |
|---|---|---|---|---|---|
| | Size | Conductivity* | Dis- | | |
| Method | (nm) | (S cm⁻¹) | persibility | Year | Ref |
| Templated | | | | | |
| Aerosol Vapor | 750 | 330 | Partially | 2019 | [12] |
| Polymerization | | | | | |
| Microemulsion | — | 165 | N/A | 2005 | [3] |
| Microemulsion | 50-55 | 100.3 | N/A | 2011 | [2] |
| | diameter | | | | |
| Microemulsion | 20-40 | 10.2 (60.5) | N/A | 2007 | [4] |
| Hallow | 600 | 1 | N/A | 2014 | [9] |
| Microspheres | | | | | |
| Microemulsion | 100 | 4 | Yes | 2008 | [10] |
| Microemulsion- | 500-800 | 3-6 | N/A | 2006 | [1] |
| Tubes | diameter | | | | |
| | 10,000 | | | | |
| | long | | | | |
| Non-Templated | | | | | |
| The Invention | 81.5 | 410 | Yes | 2023 | |
| Chemical | 50 | 85 | Yes | 2022 | [11] |
| Oxidative | | | | | |
| Polymerization | | | | | |
| Chemical | 35-120 | 50 | N/A | 2004 | [7] |
| Oxidative | | | | | |
| Polymerization | | | | | |
| Chemical | 10-20 | 6.3 | Yes** | 2011 | [8] |
| Oxidative | | | | | |
| Polymerization | | | | | |
| Chemical | 60-900 | 0.29-154 | N/A | 2013 | [5] |
| Oxidative | | | | | |
| Polymerization | | | | | |
| Miniemulsion | 35 | 0.026 | N/A | 2018 | [6] |

*Calculated using four-point probe on a compressed pellet. Values in parenthesis are of the pellets once externally doped.
**Particles were synthesized via chemical oxidative polymerization with a surfactant in an aqueous solution. The particles were used as is without purification.

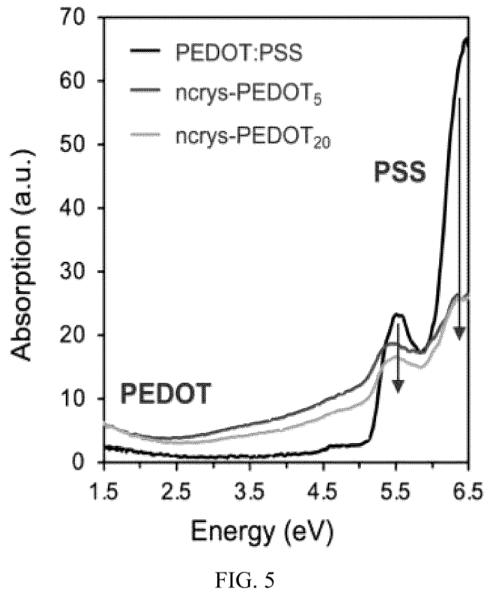

Commercial PEDOT:PSS (1-1.3 wt %) was added dropwise into a coagulation solution including both isopropanol (IPA) and sulfuric acid. Collected particles were subsequently comminuted into a fine powder using a cryogenic grinder to afford nanoparticles with an average diameter of 81.5 nm, as shown in panel c of FIG. 1. As a nonsolvent, IPA dehydrates the colloidal dispersion, enabling the NIPS phenomenon. The autoprotolysis of sulfuric acid is commonly used to stabilize the segregation of insulating PSS from the conductive PEDOT; therefore, excess PSS is selectively removed the ionomer complex, while PEDOT forms crystalline domains via π-π stacking. The degree to which PSS is lost and PEDOT crystallizes within the particles is directly related to the concentration of sulfuric acid within the coagulation bath. XPS was used to investigate the impact of the coagulation bath composition on PSS extraction. All spectra were referenced to the C is level set to 284.8 eV—analysis of the S 2p spectral region reveals the presence of PSS and PEDOT at binding energies of ≈168 and ≈164 eV, respectively, as shown in panel d of FIG. 1. A comparison of the relative intensities of these spectral components demonstrates that increasing concentrations of the acid within the coagulation bath correlates with increased PEDOT/PSS ratios; this is also demonstrated via UV/Vis analysis, as shown in FIG. 5. The XRD spectrum of the dispersible ncrys-PEDOT₅ and ncrys-PEDOT₂₀ particles represent conditions with low and high concentrations of sulfuric acid in the coagulation bath, respectively. The relative decrease in the intensity of the amorphous halo of PSS (≈2θ=16-17) further supports the loss of PSS at greater sulfuric acid concentrations. Furthermore, at the elevated acid concentration the d₍₀₁₀₎ at ≈2θ=25 shifts to a higher angle, as shown in panel e of FIG. 1, which this reflection is broad and diffuse in pristine PEDOT:PSS. This reduced d₍₀₁₀₎ spacing demonstrates a decrease in interchain π-π stacking distance of PEDOT, suggesting crystallization enhancement when coagulated with increasing sulfuric acid concentration, consistent with similar investigations of acid-crystallized PEDOT:PSS thin films. The experimental peaks were fitted to a Gaussian function and the full-width at half-maximum (FWHM) was obtained; the crystallite size was calculated using the Debye-Scherrer equation with a K-constant of 0.9. The calculations revealed that the crystallite size of the particles increases from 1.26 to 1.58 nm upon treatment with more concentrated sulfuric acid.

PEDOT:PSS films processed via acid crystallization typically display enhancements in conductivity—to investigate the conductivity of ncrys-PEDOT_{X}, disc-shaped pellets were formed by applying hydrostatic pressure to the particles and were subsequently analyzed via four-point probe measurements, as shown in panel f of FIG. 1, a typical approach to quantify conductivity of conjugated polymer-based particles. As a control, we also assessed the conductivity of lyophilized PEDOT:PSS as it is commonly used as a conductive filler for hydrogels and other composites. Both ncrys-PEDOT₅ and ncrys-PEDOT₂₀ demonstrated remarkable conductivities of 87 and 410 S cm⁻¹, values significantly larger than lyophilized PEDOT:PSS (4 S cm⁻¹). To the best of our knowledge, this is the highest reported electrical conductivity for a solid-state conducting polymer powder, as shown in panel g of FIG. 1 and Table 1. While the exact mechanism of conductivity enhancement from acid crystallization is currently under investigation, it is generally accepted that the phenomena involves the removal of PSS and enhanced crystallinity; which is consistent with the XRD, UV/Vis, XPS, and conductivity analysis of ncrys-PEDOT_{X}.

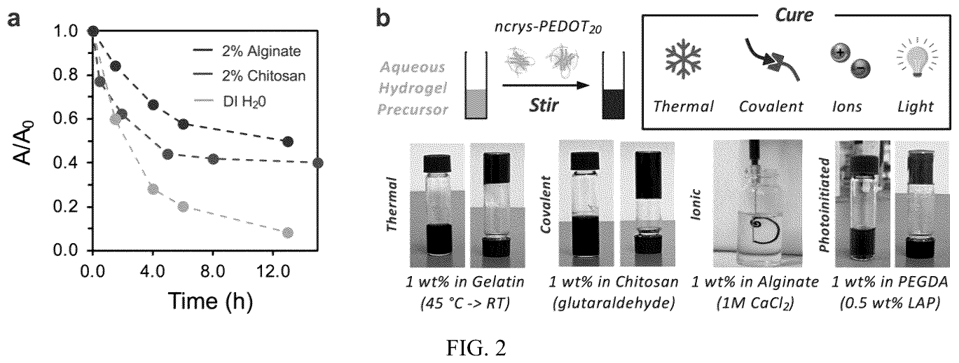

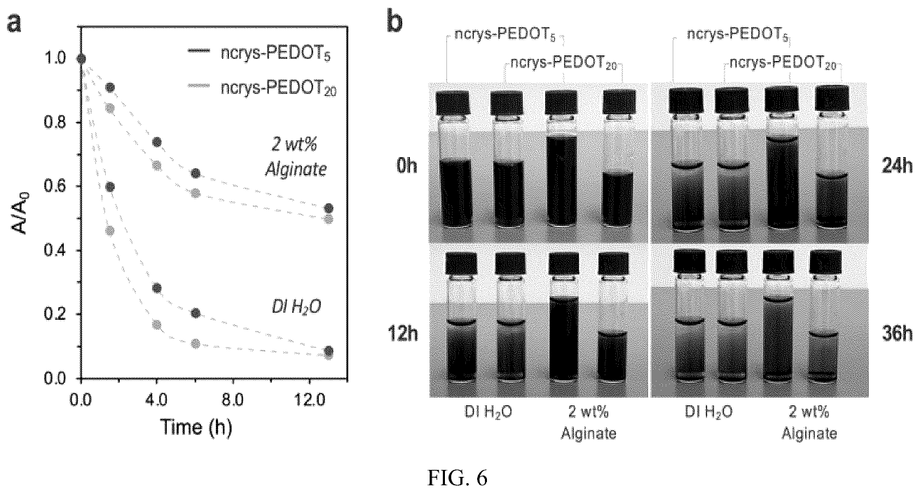

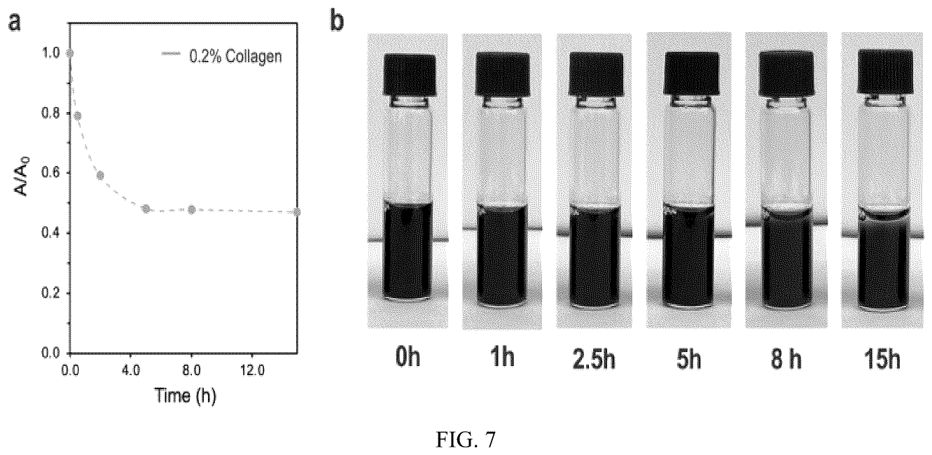

Conductive ncrys-PEDOT_{X} Incorporated Hydrogels: Conductive hydrogels are typically synthesized directly from aqueous solutions of hydrophilic natural biopolymers (alginate, gelatin, collagen, chitosan) or synthetic polymer formulations (pHEMA, PEO, PEGDA). Hydrophobic carbon-based nanomaterials such as carbon nanotubes (CNTs) and graphene (G) are common fillers for conductive hydrogels but lack stability in water, requiring complex surfactants and/or harsh oxidation to disperse, thus complicating their application within conductive hydrogels. The residual PSS within ncrys-PEDOT_{X} can act as an internal surfactant to stabilize their dispersion within aqueous biomaterial solutions, overcoming this limitation. The tunability with which this internal surfactant remains within the particle can therefore act as a handle to promote aqueous dispersibility. The kinetic stability of ncrys-PEDOT_{X} was investigated by monitoring the absorbance of 1 wt % (10 mg mL⁻¹) ncrys-PEDOT_{X} aqueous dispersions over time, a common method to profile the sedimentation kinetics of nanomaterial dispersions. Both ncrys-PEDOT₅ and ncrys-PEDOT₂₀ were first dispersed via stirring in either DI H₂O or 2% aqueous sodium alginate, the latter of which is a common precursor for conductive biomaterials, as shown in FIG. 6; such high filler concentrations are similar to those used with graphene oxide, and are orders of magnitude more concentrated than what can be achieved with untreated CNTs or G. For example, graphene nanoplatelets can only be dispersed in water without oxidation or complex surfactant optimization at concentrations less than 0.01 mg mL⁻¹. In both DI and aqueous sodium alginate solutions the sedimentation of ncrys-PEDOT₅ and ncrys-PEDOT₂₀ were similar, however ncrys-PEDOT₅ had a relatively slower rate, potentially due to the elevated amount of PSS internal surfactant. As ncrys-PEDOT₂₀ displays significantly enhanced conductivity, but with a similar aqueous sedimentation profile to ncrys-PEDOT₅, ncrys-PEDOT₂₀ was further explored in aqueous dispersions of chitosan and collagen due to their widespread use within the synthesis and design of hydrogels for biomedical applications. The particles had remarkable kinetic stability in each of the aqueous biomaterial-based hydrogel precursors—particularly considering sonication and external surfactants were not utilized, as shown in panel a of FIG. 2 and FIG. 7. Importantly the sedimentation rate is significantly slower than the rate of hydrogel crosslinking (≈0.5-1 h), allowing these particles to be directly added to aqueous hydrogel precursor solutions for conductive hydrogel formulations.

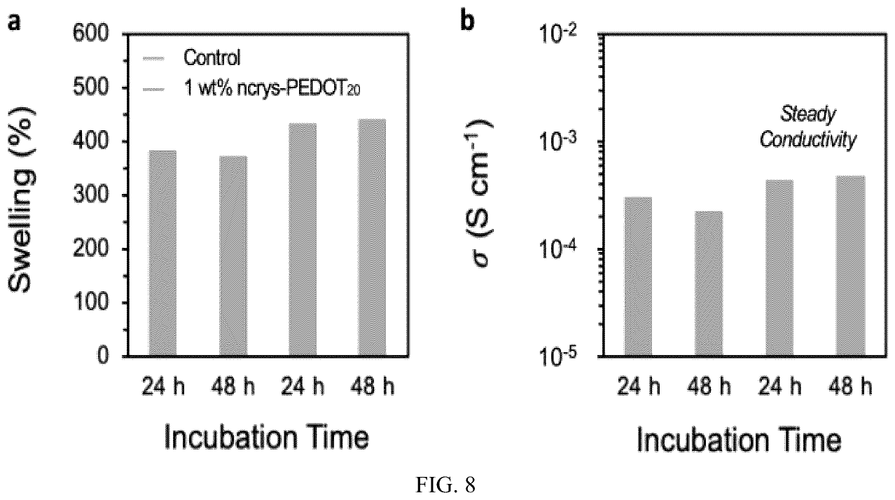

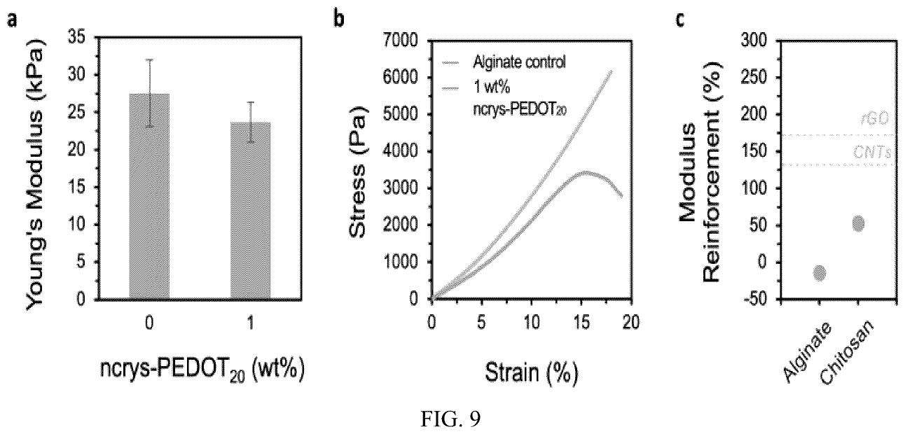

We then investigated if ncrys-PEDOT₂₀ impacted the prototypical chemistries that are utilized to crosslink biomaterials into hydrogels. Many natural aqueous biomaterial dispersions are crosslinked thermally, covalently, ionically, or via pH. Representative biomaterial dispersions of polysaccharides (agarose, chitosan), protein (gelatin), and synthetic resin (PEGDA) were loaded with 1 wt % (10 mg mL⁻¹) of ncrys-PEDOT₂₀, stirred and subsequently crosslinked, as shown in panel b of FIG. 2. Each dispersion successfully gelled demonstrating that the particles do not interfere with hydrogel-bonding networks (gelatin), Schiff base chemistry (chitosan), ionic crosslinking (alginate), or radical photopolymerization (PEGDA). Furthermore, each hydrogel crosslinked rapidly and demonstrated no evidence of sedimentation—this suggests compatibility of ncrys-PEDOT₂₀ with the common modalities used throughout biomaterial/hydrogel chemistry. Analysis of the swelling and conductivity of the alginate hydrogel with 1 wt % ncrys-PEDOT₂₀ suggests the particles do not have a deleterious impact on hydrogel stability, as shown in FIG. 8, however such affects are anticipated to be highly dependent on the hydrogel composition and are a topic for future investigation. As the loading of conductive fillers can increase the modulus (i.e., stiffness) of the hydrogel, we screened several hydrogels to benchmark the modulus reinforcement of ncrys-PEDOT₂₀ compared to other common fillers. The modulus reinforcement is dependent on polymer-particle interactions, therefore the impact ncrys-PEDOT_{X} has on a hydrogel will be dependent on several factors (polymer structure, polymer loading, crosslinker structure, crosslinker loading, additives, type of ncrys-PEDOT_{X}, etc.), as such it is challenging to make generalizable claims. As a representative example, mechanical analysis (DMA) of the alginate hydrogel demonstrates a slight decrease in the Young's modulus of the hydrogel with ncrys-PEDOT₂₀ incorporation however both hydrogels feature similar stiffness (i.e., soft tissue) to the prior report of the alginate control, as shown in FIG. 9. The slight decrease in the modulus of the alginate hydrogel may suggest the particles reduce the crosslink density as the ionic nature of the calcium crosslinking is weak. This is supported by the slight increase in the modulus of the chitosan hydrogels with particle incorporation—these hydrogels are covalently crosslinked. At 1 wt % loading of ncrys-PEDOT₂₀, each of the tested gels demonstrates substantially less modulus reinforcement than what is seen on average with CNTs (130%) or reduced graphene oxide (170%). Recent work with hydrophilic CPs has suggested that hygroscopic side chains can reduce modulus reinforcement; the internal PSS surfactant may behave similarly, preventing detrimental stiffening upon loading. As the impact of ncrys-PEDOT_{X} on stiffness is hydrogel dependent, future application of the particles should also investigate reinforcement behavior.

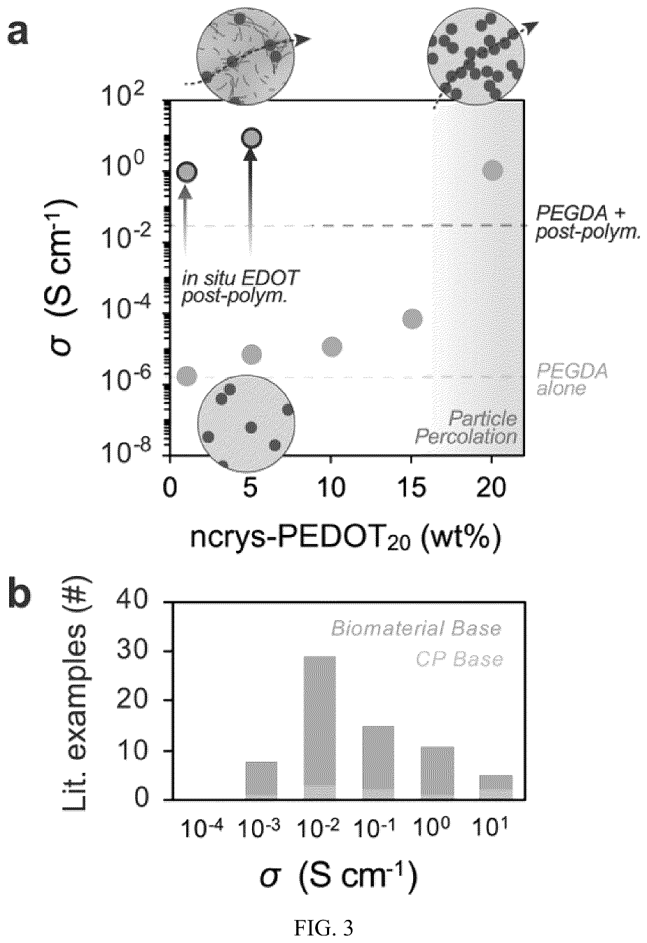

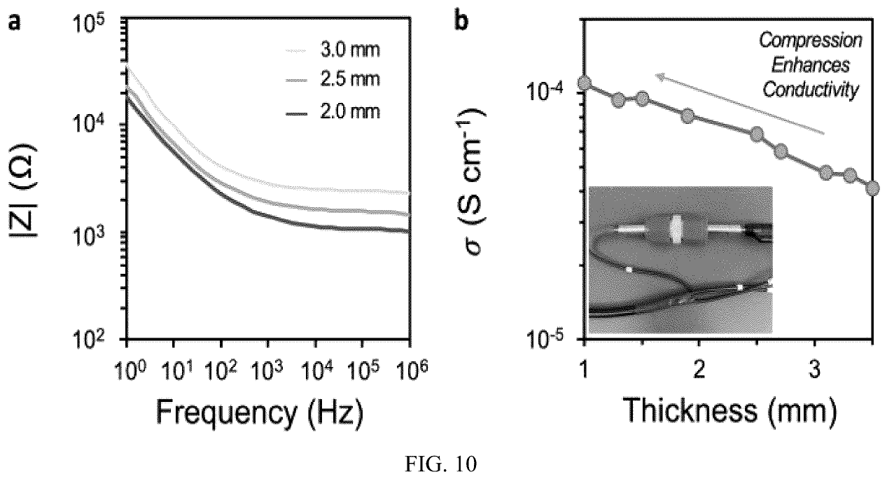

For bioelectronic applications conductive hydrogels require sufficient electronic conductivity to facilitate the transmission of endogenous or exogenous bioelectricity throughout the material, requiring 3D charge percolation. Prior experiments have suggested that as the loading of conducting filler is increased, a dramatic enhancement in conductivity is observed which is attributed to the formation of percolation pathways for charge carriers. The filler volume required to achieve percolation is related to the aspect ratio; for fillers of roughly spherical shape, similar size, and random orientation, a loading of 16% has been found—known as the Sher-Zallen invariant. While 1D and 2D materials such as CNTs or G would require reduced loadings to achieve percolation, these materials have limited aqueous dispersibility and the relatively high surface area of their form factor compared to spherical particles promotes aggregation. As high loadings of conductive filler are typically incompatible with hydrophilic hydrogel precursors, this challenge has been circumvented by processing hydrogels directly out of conjugated polymers, typically PEDOT:PSS. However, many applications require the chemical and mechanical features of hydrogels with a natural or synthetic biomaterial base, rather than a CP base to facilitate biocompatibility and biological outcomes such as cellular differentiation. To observe the endowed percolation behavior of ncrys-PEDOT₂₀ within a prototypical hydrogel, particles were systematically loaded within the aqueous PEGDA resin—this resin was chosen due to its widespread use for 3D tissue engineering constructs, biosensing media, and drug-controlled release matrices. As anticipated, a significant increase in conductivity was observed between 15% and 20% incorporation, with the highest loading displaying a remarkable conductivity of 1.1 S cm⁻¹, as shown in panel a of FIG. 3. As a loading of 20% or more of acid-crystallized CP particles may negate the desired properties of the composite hydrogel, we also explored a postpolymerization strategy to achieve percolative electronic transport with significantly lower particle loading. PEGDA hydrogels with 1 and 5 wt % loadings show significant enhancements in conductivity upon subsequent in situ polymerization of EDOT. With this approach, we achieve 8.2 S cm⁻¹ with 5% particle loading, which is notably >400 fold higher than an EDOT postpolymerization in PEDGA alone (0.02 S cm⁻¹). It should be noted that the use of EDOT (as opposed to derivatives thereof), and its particular polymerization and loading conditions is shown as a proof of concept and is the subject of future investigation. Furthermore, extrinsic factors such as applied stress can impact the conductivity of the hydrogels due to changes in interparticle distance, as shown in FIG. 10. A survey of literature examples reporting conductive hydrogels derived from 1) a CP base or 2) a biomaterial base with loaded conductive filler demonstrates that the afforded hydrogel has remarkable conductivity, well beyond the typical range of 10⁻³-10⁻¹ S cm⁻¹, as shown in panel b of FIG. 3 and Table 2.

TABLE 2
Literature Comparison of Conductive Hydrogels

| | Conductivity* | | | Conductivity* | |
|---|---|---|---|---|---|
| Hydrogel Base | (S cm⁻¹) | Ref | Hydrogel Base | (S cm⁻¹) | Ref |
| Conjugated Polymer* | | | | | |
| PEDOT:PSS | 47.4 | [13] | PEDOT:PSS | 0.1 | [20] |
| PEDOT:PSS | 38 | [14] | PEDOT:PSS | 0.087 | [21] |
| PEDOT:PSS | 14 | [15] | PPy | 0.005 | [22] |
| PEDOT:PSS | 8.8 | [16] | PEDOT | 0.0041 | [23] |
| PPy-PEDOT:PSS | 8.67 | [17] | PEDOT:PSS | 0.00118 | [24] |
| PEDOT:PSS | 0.23 | [18] | PEDOT:PSS | 0.00014 | [25] |
| PANI | 0.11 | [19] | | | |
| Biomaterial** | | | | | |
| PPEGMA-PAA | 4.3 | [26] | aCD-PNIPAM | 0.0064 | [53] |
| Chitosan-Gelatin | 0.622 | [27] | Alginate | 0.00633 | [54] |
| PEI-PAAM | 0.61 | [28] | Chitosan | 0.00468 | [55] |
| Collagen | 0.45 | [29] | Chitosan | 0.0044 | [56] |
| PAAM | 0.26 | [30] | PAAM-PEG | 0.0038 | [57] |
| Agarose | 0.195 | [31] | Collagen- | 0.0035 | [58] |
| | | | Alginate | | |
| Tannic Acid | 0.18 | [32] | MA-Collagen | 0.0034 | [59] |
| Polylactic Acid | 0.146 | [33] | PEG | 0.00289 | [60] |
| PSS | 0.13 | [34] | PAAM | 0.0026 | [61] |
| Gellan Gum | 0.107 | [35] | Chitosan-PEG | 0.00242 | [62] |
| Polyvinyl Alcohol | 0.1 | [36] | Guar Slime | 0.00222 | [63] |
| Cellulose | 0.08 | [37] | PAMPS-PAAM | 0.0016 | [64] |
| Chitosan-Gelatin | 0.0768 | [38] | Agarose-PAM- | 0.0015 | [65] |
| | | | PVA | | |
| Alginate | 0.06 | [39] | PAA | 0.00141 | [66] |
| Chitosan | 0.048 | [40] | PAA | 0.0012 | [67] |
| TOCNF-PAA | 0.039 | [41] | HA | 0.00118 | [68] |
| Collagen-HA | 0.03705 | [42 | Silk Fibroin | 0.00114 | [69] |
| HA-Chitosan | 0.031 | [43] | PNIPAM | 0.0008 | [70] |
| Gelatin-PAAM | 0.0307 | [44] | PPAM | 0.0006 | [71] |
| HA-Chitosan | 0.0275 | [43] | Chitosan | 0.00028 | [72] |
| PNAGA-PAMPS | 0.022 | [45] | PAAM | 0.00014 | [73] |
| GeIMA | 0.0135 | [46] | GelMa | 0.000134 | [74] |
| y-polyglutamic acid | 0.013 | [47] | Alginate | 0.00011 | [75] |
| Alginate-Gelatin | 0.012 | [48] | Alginate | 0.000079 | [76] |
| HA-Chitosan- | 0.00948 | [49] | Dextran | 0.000034 | [77] |
| Gelatin | | | | | |
| HA-Gelatin- | 0.0092 | [50] | Gelatin-BaG | 0.00000200 | [78] |
| Alginate | | | | | |
| HA | 0.0073 | [51] | Gelatin | 0.000001 | [79] |
| Gellan Gum | 0.007 | [52] | Chitosan | 0.000001000 | [80] |

*Hydrogels were made primarily from the conjugated polymer. Additives (solvent, acid, etc.), ice templating, crosslinking, and other processing methods are used to gel the conjugated polymer base.
** Hydrogels are made from natural or synthetic biomaterials. Conductive fillers (PEDOT, PPy, PEDOT:PSSS, PANI, etc.) are added to the biomaterial base.

3D Printing of ncrys-PEDOT_{X} Incorporated Resins: 3D printing has matured into a promising technique for rapidly generating soft matter with complex form factors for biomedical applications, such as stents, scaffolds for tissue regeneration, implants, and soft robotics, among others. Printing conductive hydrogels while maintaining structural control of fine (<200 μm) features remains a challenge, as the incorporation of electroactive fillers into printable resins can dramatically impact the rate of photopolymerization or clog the printing nozzle due to particle aggregation during the printing process—arising from poor particle dispersibility. Alternatively, 3D printed insulating hydrogels can be endowed with electrical conductivity through 1) soaking the part in monomer solution, and 2) subsequently polymerizing the monomer in situ to generate a double network hydrogel—however such systems have limited control of the final CP loading, and can lack homogenous distribution of incorporated CP. While a growing literature has adopted making 3D printable resins directly from PEDOT:PSS, many biomedical applications require the high water content, mechanical properties, and biocompatibility of widely utilized synthetic biomaterials which mimic the native extracellular matrix microenvironments. It would be attractive to simply add a dispersible conductive filler to a pre-existing aqueous resin to directly print conductive hydrogels.

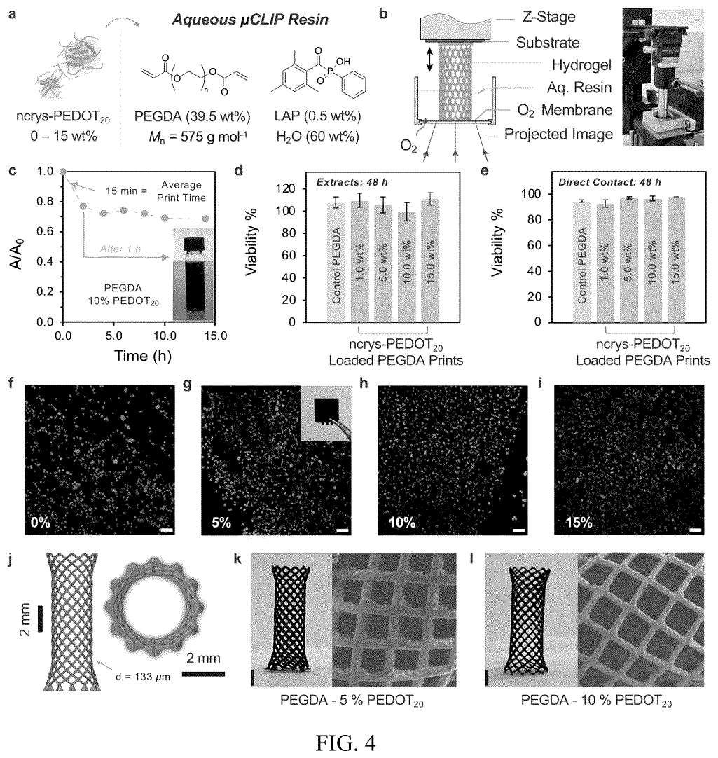

An aqueous photoprintable conductive resin was formulated through the addition of ncrys-PEDOT₂₀ into a mixture of poly(ethylene glycol)diacrylate (PEGDA) and lithium phenyl (2,4,6-trimethylbenzoyl) phosphinate (LAP) photoinitiator in water (panel a of FIG. 4). The aqueous PEGDA resin was utilized to demonstrate the printability of ncrys-PEDOT_{X} due to its widespread adoption for 3D printing and its noncytotoxic nature; ncrys-PEDOT₂₀ was used as the filler as it displayed the highest conductivity of the particles investigated while maintaining dispersibility. Microcontinuous liquid interface production (μCLIP) projection stereolithography, was used to rapidly print conductive hydrogels with the ncrys-PEDOT₂₀ incorporated at loadings of 1, 5, 10, and 15 wt %, as shown in panel b of FIG. 4. The kinetic stability of ncrys-PEDOT₂₀ within the resin was investigated via sedimentation; a high loading of 10 wt % ncrys-PEDOT₂₀ was loaded into the aqueous resin simply through vortexing. A remarkable kinetic stability was observed without any sonication or added surfactants, as shown in panel c of FIG. 4; potentially due to the enhanced viscosity of the resin compared to the biomaterial dispersions, as shown in panel a of FIG. 2. As the average print time of a part is 15 min, and the part is printed from the bottom of the bath, the particles remain dispersed throughout the μCLIP 3D printing process.

Biocompatibility is an important and often overlooked property of materials intended for bioelectronic applications. The biocompatibility of ncrys-PEDOT₂₀ was assessed by proxy of particle-loaded 3D printed PEGDA gels. Following a modified ISO 10993-5 protocol, in vitro cytotoxicity tests were performed both on extracts from the particle-loaded hydrogels and through direct contact via alamarBlue (panel d of FIG. 4) and live/dead (panel e of FIG. 4) assays, respectively. Both assays confirmed high cell viability (>95%) at all loadings up to 15% of ncrys-PEDOT₂₀. Significant differences in viability were not observed between the PEGDA hydrogels with and without particle incorporation, therefore the particles appear to be biocompatible. Imaging from the live/dead analysis (panels f-i of FIG. 4) demonstrates slight enhancements in cell spreading/adhesion at increased loadings of the ncrys-PEDOT₂₀ particles. Further, hemolysis testing on isolated ncrys-PEDOT₂₀ particles suggests high blood compatibility (5.77% hemolysis), an important material requirement for bioelectronic applications such as regenerative engineering or healthcare monitoring. The high compatibility and positive cell outcomes in the presence of the acid-crystallized PEDOT particles are consistent with other observations of PEDOT:PSS incorporated systems.

To demonstrate the capability to print complex, biomedically relevant geometries, particle incorporated aqueous PEGDA resins were printed. Complex tubular scaffolds resembling esophageal stents with 133.33 μm diameter struts were printed (panel j of FIG. 4). Stents with both 5% (panel k of FIG. 4) and 10% particle loading (panel 1 of FIG. 4) demonstrated high structural fidelity with complex, small (<150 μm) features. Such fine features are extraordinary considering the relatively high loading of conductive filler compared to similar formulations, and that both sonication and surfactants were not required for resin preparation. As the dark ncrys-PEDOT₂₀ particles did not interfere with the photopolymerization process, acid-crystallized PEDOT particles can be incorporated within other biomedical resin formations and printed for a wide variety of applications such as biological signal recording, strain sensing, stimulation electrodes, drug delivery, and scaffolds for tissue regeneration.

### CONCLUSION

A simple, scalable acid-based nonsolvent induced phase separation (NIPS) approach was leveraged to afford acid crystalized PEDOT nanoparticles (ncrys-PEDOT_{X}) from commercial PEDOT:PSS with dispersibility arising from an internal surfactant. By tuning the volume ratio of sulfuric acid within the coagulation bath, the PEDOT/PSS ratio was optimized to afford particles with conductivity rivaling all reports of conjugated polymer particles to date (σ_{ncrys-PEDOT20}=410 S cm⁻¹). Evidence from spectroscopy and scattering suggest that both the removal of insulating PSS and a decrease in the interchain π-π stacking distance of PEDOT lead to enhanced conductivity within the crystallized particles, similar to reports of acid crystalized thin films of PEDOT:PSS, but in solution. Sedimentation and conductivity analyses suggests there is a fine balance when removing PSS via acid crystallization to achieve enhanced conductivity, while maintaining dispersibility.

When incorporated within aqueous dispersions of both natural (polysaccharides and proteins) and synthetic biomaterials, ncrys-PEDOT₂₀ demonstrated high compatibility without the need of external surfactants or sonication—this was observed at concentrations orders of magnitude higher than what is possible with pure graphene or carbon nanotubes. Furthermore, the particles did not interfere with the toolbox of chemistries typically used to crosslink hydrogels (hydrogen bonding, ionic bonding, Schiff-base chemistry, and radical photopolymerization). By directly adding ncrys-PEDOT₂₀ to common hydrogel formulations, highly conductive composites were achieved with a percolation threshold between 15 and 20 wt % loading. The observed conductivity (>1 S cm⁻¹) is remarkable considering the base of the hydrogel was not derived from a CP. Given the aqueous dispersibility and kinetic stability at high loadings (100 mg mL⁻¹), the ncrys-PEDOT₂₀ incorporated PEGDA resin was utilized for microstereolithography. The afforded hydrogels demonstrated biocompatibility and structural fidelity with complex, small (<150 μm) features. Such fine features are incredible considering the high loading of conductive filler, and that both sonication and surfactants were not required for resin preparation. As the reported particles display enhanced aqueous dispersibility, high conductivity, biocompatibility, and scalable access from a common commercial ink (PEDOT:PSS), we anticipate ncrys-PEDOT_{X} offers a robust platform toward conductive hydrogels for TERM and other bioelectronic applications. Unlike other conductive hydrogel formulations which require complex multistep procedures and additives, ncrys-PEDOT_{X} can be directly added to endow already existing and commonplace hydrogel formulations for biomedical applications with conductivity.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### LISTING OF REFERENCES

[1]. Zhang X, et al. Chemical Synthesis of PEDOT Nanotubes. Macromolecules 39, 470-472 (2006).
[2]. Zheng H, Jiang Y, Xu J, Yang Y. Analysis on the characteristic properties of PEDOT nano-particle based on reversed micelle method. J Wuhan Univ Technol-Mat Sci Edit 26, 422-428 (2011).
[3]. Lei Y, Oohata H, Kuroda S-i, Sasaki S, Yamamoto T. Highly electrically conductive poly(3,4-ethylenedioxythiophene) prepared via high-concentration emulsion polymerization. Synth Met 149, 211-217 (2005).
[4]. Yang Y, Jiang Y, Xu J, Yu J. Conducting polymeric nanoparticles synthesized in reverse micelles and their gas sensitivity based on quartz crystal microbalance. Polymer 48, 4459-4465 (2007).
[5]. Paradee N, Sirivat A. Synthesis of poly(3,4-ethylenedioxythiophene) nanoparticles via chemical oxidation polymerization. Polym Int 63, 106-113 (2014).
[6]. Culebras M, et al. Conducting PEDOT Nanoparticles: Controlling Colloidal Stability and Electrical Properties. J Phys Chem C 122, 19197-19203 (2018).
[7]. Choi J W, Han M G, Kim S Y, Oh S G, Im S S. Poly(3,4-ethylenedioxythiophene) nanoparticles prepared in aqueous DBSA solutions. Synth Met 141, 293-299 (2004).
[8]. Wu C-H, Don T-M, Chiu W-Y. Characterization and conversion determination of stable PEDOT latex nanoparticles synthesized by emulsion polymerization. Polymer 52, 1375-1384 (2011).
[9]. Pang R, et al. Preparation of multi-shelled conductive polymer hollow microspheres by using Fe₃O₄ hollow spheres as sacrificial templates. Chem Commun 50, 12493-12496 (2014).
[10]. Dai C-a, Chang C-j, Chi H-y, Chien H-t, Su W-f, Chiu W-y. Emulsion synthesis of nanoparticles containing PEDOT using conducting polymeric surfactant: Synergy for colloid stability and intercalation doping. J Polym Sci A Polym Chem 46, 2536-2548 (2008).
[11]. Cao G, Cai S, Chen Y, Zhou D, Zhang H, Tian Y. Facile synthesis of highly conductive and dispersible PEDOT particles. Polymer 252, 124952 (2022).
[12]. Lu Y, et al. Synthesis of Submicron PEDOT Particles of High Electrical Conductivity via Continuous Aerosol Vapor Polymerization. ACS Appl Mater Interfaces 11, 47320-47329 (2019).
[13]. Liu Y, et al. Soft and elastic hydrogel-based microelectronics for localized low-voltage neuromodulation. Nat Biomed Eng 3, 58-68 (2019).
[14]. Lu B, et al. Pure PEDOT:PSS hydrogels. Nat Commun 10, 1043 (2019).
[15]. Chen F-J, et al. Rational design of a highly porous electronic scaffold with concurrent enhancement in cell behaviors and differentiation under electrical stimulation. J Mater Chem B 9, 7674-7685 (2021).
[16]. Yao B, et al. Ultrahigh-Conductivity Polymer Hydrogels with Arbitrary Structures. Adv Mater 29, 1700974 (2017).
[17]. Ren X, et al. Highly Conductive PPy-PEDOT:PSS Hybrid Hydrogel with Superior Biocompatibility for Bioelectronics Application. ACS Appl Mater Interfaces 13, 25374-25382 (2021).
[18]. Feig V R, Tran H, Lee M, Bao Z. Mechanically tunable conductive interpenetrating network hydrogels that mimic the elastic moduli of biological tissue. Nat Commun 9, 2740 (2018).
[19]. Pan L, et al. Hierarchical nanostructured conducting polymer hydrogel with high electrochemical activity. Proceedings of the National Academy of Sciences 109, 9287-9292 (2012).
[20]. Zhang S, et al. Room-Temperature-Formed PEDOT:PSS Hydrogels Enable Injectable, Soft, and Healable Organic Bioelectronics. Adv Mater 32, 1904752 (2020).
[21]. Dai T, Shi Z, Shen C, Wang J, Lu Y. Self-strengthened conducting polymer hydrogels. Synth Met 160, 1101-1106 (2010).
[22]. Lu Y, et al. Elastic, Conductive, Polymeric Hydrogels and Sponges. Sci Rep 4, 5792 (2014).
[23]. Dai T, Jiang X, Hua S, Wang X, Lu Y. Facile fabrication of conducting polymer hydrogels via supramolecular self-assembly. Chem Commun, 4279-4281 (2008).
[24]. Solazzo M, Monaghan M G. Structural crystallisation of crosslinked 3D PEDOT:PSS anisotropic porous biomaterials to generate highly conductive platforms for tissue engineering applications. Biomater Sci 9, 4317-4328 (2021).
[25]. Guex A G, et al. Highly porous scaffolds of PEDOT:PSS for bone tissue engineering. Acta Biomater 62, 91-101 (2017).
[26]. Naficy S, Razal J M, Spinks G M, Wallace G G, Whitten P G. Electrically Conductive, Tough Hydrogels with pH Sensitivity. Chem Mater 24, 3425-3433 (2012).
[27]. Wang S, et al. Chitosan/gelatin porous scaffolds assembled with conductive poly(3,4-ethylenedioxythiophene) nanoparticles for neural tissue engineering. J Mater Chem B 5, 4774-4788 (2017).
[28]. Li Y, et al. Carboxymethyl cellulose assisted polyaniline in conductive hydrogels for high-performance self-powered strain sensors. Carbohydr Polym 298, 120060 (2022).
[29]. Li Q, Yue C, Chen T, Ding C, Zhang H. Construction and characterization of conductive collagen/multiwalled carbon nanotube composite films for nerve tissue engineering. AIP Adv 12, (2022).
[30]. Tondera C, et al. Highly Conductive, Stretchable, and Cell-Adhesive Hydrogel by Nanoclay Doping. Small 15, 1901406 (2019).
[31]. Hur J, et al. Polypyrrole/Agarose-Based Electronically Conductive and Reversibly Restorable Hydrogel. ACS Nano 8, 10066-10076 (2014).
[32]. Zhou L, et al. Soft Conducting Polymer Hydrogels Cross-Linked and Doped by Tannic Acid for Spinal Cord Injury Repair. ACS Nano 12, 10957-10967 (2018).
[33]. Liu R, et al. Electrical stimulation mediated the neurite outgrowth of PC-12 cells on the conductive polylactic acid/reduced graphene oxide/polypyrrole composite nanofibers. Appl Surf Sci 560, 149965 (2021).
[34]. Chen J, Peng Q, Thundat T, Zeng H. Stretchable, Injectable, and Self-Healing Conductive Hydrogel Enabled by Multiple Hydrogen Bonding toward Wearable Electronics. Chem Mater 31, 4553-4563 (2019).
[35]. Warren H, in het Panhuis M. Highly conducting composite hydrogels from gellan gum, PEDOT:PSS and carbon nanofibres. Synth Met 206, 61-65 (2015).
[36]. Li W, Gao F, Wang X, Zhang N, Ma M. Strong and Robust Polyaniline-Based Supramolecular Hydrogels for Flexible Supercapacitors. Angew Chem Int Ed 55, 9196-9201 (2016).
[37]. Shi Z, et al. In Situ Synthesis of Robust Conductive Cellulose/Polypyrrole Composite Aerogels and Their Potential Application in Nerve Regeneration. Angew Chem Int Ed 53, 5380-5384 (2014).
[38]. Wang S, et al. 3D culture of neural stem cells within conductive PEDOT layer-assembled chitosan/gelatin scaffolds for neural tissue engineering. Mater Sci Eng C 93, 890-901 (2018).
[39]. Yang B, et al. A conductive PEDOT/alginate porous scaffold as a platform to modulate the biological behaviors of brown adipose-derived stem cells. Biomater Sci 8, 3173-3185 (2020).
[40]. Du P, Wang J, Hsu Y-I, Uyama H. Bio-Inspired Homogeneous Conductive Hydrogel with Flexibility and Adhesiveness for Information Transmission and Sign Language Recognition. ACS Appl Mater Interfaces 15, 23711-23724 (2023).
[41]. Chen Y, et al. A Skin-Inspired Stretchable, Self-Healing and Electro-Conductive Hydrogel with A Synergistic Triple Network for Wearable Strain Sensors Applied in Human-Motion Detection. Nanomaterials 9, (2019).
[42]. Lin X, et al. Antibacterial Conductive Collagen-Based Hydrogels for Accelerated Full-Thickness Wound Healing. ACS Appl Mater Interfaces 15, 22817-22829 (2023).
[43]. Xu Y, et al. Cytocompatible, Injectable, and Electroconductive Soft Adhesives with Hybrid Covalent/Noncovalent Dynamic Network. Adv Sci 6, 1802077 (2019).
[44]. Li Y, et al. A stretchable and self-healable conductive hydrogels based on gelation/polyacrylamide/polypyrrole for all-in-one flexible supercapacitors with high capacitance. Colloids Surf 636, 128145 (2022).
[45]. Wu Q, et al. A robust, highly stretchable supramolecular polymer conductive hydrogel with self-healability and thermo-processability. Sci Rep 7, 41566 (2017).
[46]. Dutta S D, Ganguly K, Randhawa A, Patil T V, Patel D K, Lim K-T. Electrically stimulated 3D bioprinting of gelatin-polypyrrole hydrogel with dynamic semi-IPN network induces osteogenesis via collective signaling and immunopolarization. Biomaterials 294, 121999 (2023).
[47]. Zhang C, et al. Highly adhesive and self-healing y-PGA/PEDOT:PSS conductive hydrogels enabled by multiple hydrogen bonding for wearable electronics. Nano Energy 95, 106991 (2022).
[48]. Distler T, et al. Electrically Conductive and 3D-Printable Oxidized Alginate-Gelatin Polypyrrole:PSS Hydrogels for Tissue Engineering. Adv Healthcare Mater 10, 2001876 (2021).
[49]. Wang S, Guan S, Zhu Z, Li W, Liu T, Ma X. Hyaluronic acid doped-poly(3,4-ethylenedioxythiophene)/chitosan/gelatin (PEDOT-HA/Cs/Gel) porous conductive scaffold for nerve regeneration. Mater Sci Eng C 71, 308-316 (2017).
[50]. Aycan D, Dolapgi N, Karaca O G, Alemdar N. Polysaccharide-based electroconductive films for controlled release of ciprofloxacin. J Appl Polym Sci 139, e52761 (2022).
[51]. Yang J, Choe G, Yang S, Jo H, Lee J Y. Polypyrrole-incorporated conductive hyaluronic acid hydrogels. Biomater Res 20, 31 (2016).
[52]. Warren H, Gately R D, O'Brien P, Gorkin Iii R, in het Panhuis M. Electrical conductivity, impedance, and percolation behavior of carbon nanofiber and carbon nanotube containing gellan gum hydrogels. J Polym Sci Part B: Polym Phys, 52, 864871 (2014).
[53]. Zhu Y, Liu S, Shi X, Han D, Liang F. A thermally responsive host-guest conductive hydrogel with self-healing properties. Mater Chem Front 2, 2212-2219 (2018).
[54]. Wright C J, et al. Synthesis and 3D Printing of Conducting Alginate-Polypyrrole Ionomers. Gels 6, (2020).
[55]. Xu C, et al. Biodegradable and electroconductive poly(3,4-ethylenedioxythiophene)/carboxymethyl chitosan hydrogels for neural tissue engineering. Mater Sci Eng C 84, 32-43 (2018).
[56]. Kashi M, Baghbani F, Moztarzadeh F, Mobasheri H, Kowsari E. Green synthesis of degradable conductive thermosensitive oligopyrrole/chitosan hydrogel intended for cartilage tissue engineering. Int J Biol Macromol 107, 1567-1575 (2018).
[57]. Chen P-W, Ji D-H, Zhang Y-S, Lee C, Yeh M-Y. Electroactive and Stretchable Hydrogels of 3,4-Ethylenedioxythiophene/thiophene Copolymers. ACS Omega 8, 6753-6761 (2023).
[58]. Roshanbinfar K, et al. Electroconductive Biohybrid Hydrogel for Enhanced Maturation and Beating Properties of Engineered Cardiac Tissues. Adv Funct Mater 28, 1803951 (2018).
[59]. Ravichandran R, Martinez J G, Jager E W H, Phopase J, Turner A P F. Type I Collagen-Derived Injectable Conductive Hydrogel Scaffolds as Glucose Sensors. ACS Appl Mater Interfaces 10, 16244-16249 (2018).
[60]. Gong H Y, Park J, Kim W, Kim J, Lee J Y, Koh W-G. A Novel Conductive and Micropatterned PEG-Based Hydrogel Enabling the Topographical and Electrical Stimulation of Myoblasts. ACS Appl Mater Interfaces 11, 47695-47706 (2019).
[61]. Dai T, Qing X, Zhou H, Shen C, Wang J, Lu Y. Mechanically strong conducting hydrogels with special double-network structure. Synth Met 160, 791-796 (2010).
[62]. Dong R, Zhao X, Guo B, Ma P X. Self-Healing Conductive Injectable Hydrogels with Antibacterial Activity as Cell Delivery Carrier for Cardiac Cell Therapy. ACS Appl Mater Interfaces 8, 17138-17150 (2016).
[63]. Li S, Wang L, Zheng W, Yang G, Jiang X. Rapid Fabrication of Self-Healing, Conductive, and Injectable Gel as Dressings for Healing Wounds in Stretchable Parts of the Body. Adv Funct Mater 30, 2002370 (2020).
[64]. Kishi R, et al. Electro-conductive double-network hydrogels. J Polym Sci B Polym Phys 50, 790-796 (2012).
[65]. Azar M G, et al. Tough and flexible conductive triple network hydrogels based on agarose/polyacrylamide/polyvinyl alcohol and poly(3,4-ethylenedioxythiophene):polystyrene sulfonate. Polym Int 70, 1523-1533 (2021).
[66]. Hosseinzadeh S, Rezayat S M, Vashegani-Farahani E, Mahmoudifard M, Zamanlui S, Soleimani M. Nanofibrous hydrogel with stable electrical conductivity for biological applications. Polymer 97, 205-216 (2016).
[67]. Dai T, Qing X, Lu Y, Xia Y. Conducting hydrogels with enhanced mechanical strength. Polymer 50, 5236-5241 (2009).
[68]. Shi M, Dong R, Hu J, Guo B. Conductive self-healing biodegradable hydrogel based on hyaluronic acid-grafted-polyaniline as cell recruitment niches and cell delivery carrier for myogenic differentiation and skeletal muscle regeneration. Chem Eng J 457, 141110 (2023).
[69]. Zhao Y, Liang Y, Ding S, Zhang K, Mao H-q, Yang Y. Application of conductive PPy/SF composite scaffold and electrical stimulation for neural tissue engineering. Biomaterials 255, 120164 (2020).
[70]. Zhang H, Yue M, Wang T, Wang J, Wu X, Yang S. Conductive hydrogel-based flexible strain sensors with superior chemical stability and stretchability for mechanical sensing in corrosive solvents. New J Chem 45, 4647-4657 (2021).
[71]. Sun F, et al. Highly transparent, adhesive, stretchable and conductive PEDOT:PSS/polyacrylamide hydrogels for flexible strain sensors. Colloids Surf 625, 126897 (2021).
[72]. Huang L, et al. Biocompatible Chitin Hydrogel Incorporated with PEDOT Nanoparticles for Peripheral Nerve Repair. ACS Appl Mater Interfaces 13, 16106-16117 (2021).
[73]. Jo H, et al. Electrically conductive graphene/polyacrylamide hydrogels produced by mild chemical reduction for enhanced myoblast growth and differentiation. Acta Biomater 48, 100-109 (2017).
[74]. Zhang Y, et al. Conductive GelMA/PEDOT: PSS Hybrid Hydrogel as a Neural Stem Cell Niche for Treating Cerebral Ischemia-Reperfusion Injury. Front Mater 9, (2022).
[75]. Yang S, et al. Polypyrrole/Alginate Hybrid Hydrogels: Electrically Conductive and Soft Biomaterials for Human Mesenchymal Stem Cell Culture and Potential Neural Tissue Engineering Applications. Macromol Biosci 16, 1653-1661 (2016).
[76]. Babeli I, Ruano G, Casanovas J, Ginebra M-P, Garcia-Torres J, Alemin C. Conductive, self-healable and reusable poly(3,4-ethylenedioxythiophene)-based hydrogels for highly sensitive pressure arrays. J Mater Chem C 8, 8654-8667 (2020).
[77]. Guo B, Qu J, Zhao X, Zhang M. Degradable conductive self-healing hydrogels based on dextran-graft-tetraaniline and N-carboxyethyl chitosan as injectable carriers for myoblast cell therapy and muscle regeneration. Acta Biomater 84, 180-193 (2019).
[78]. Yazdimamaghani M, Razavi M, Mozafari M, Vashaee D, Kotturi H, Tayebi L. Biomineralization and biocompatibility studies of bone conductive scaffolds containing poly(3,4-ethylenedioxythiophene):poly(4-styrene sulfonate) (PEDOT:PSS). J Mater Sci: Mater Med 26, 274 (2015).
[79]. Furlani F, et al. Electroconductive and injectable hydrogels based on gelatin and PEDOT:PSS for a minimally invasive approach in nervous tissue regeneration. Biomater Sci 10, 2040-2053 (2022).
[80]. Au-Yong S, et al. Electrochemically Enhanced Delivery of Pemetrexed from Electroactive Hydrogels. Polymers 14, (2022).
[81]. J. Rivnay, R. M. Owens, G. G. Malliaras, Chem. Mater. 2014, 26, 679.
[82]. A. J. Petty I I, R. L. Keate, B. Jiang, G. A. Ameer, J. Rivnay, Chem. Mater. 2020, 32, 4095.
[83]. S. Inal, J. Rivnay, A.-O. Suiu, G. G. Malliaras, I. McCulloch, Acc. Chem. Res. 2018, 51, 1368.
[84]. B. D. Paulsen, K. Tybrandt, E. Stavrinidou, J. Rivnay, Nat. Mater. 2020, 19, 13.
[85]. G. Zhao, H. Zhou, G. Jin, B. Jin, S. Geng, Z. Luo, Z. Ge, F. Xu, Prog. Polym. Sci. 2022, 131, 101573.
[86]. J. Tropp, J. Rivnay, J. Mater. Chem. C 2021, 9, 13543.
[87]. Z. Peng, L. Ye, H. Ade, Mater. Horiz. 2022, 9, 577.
[88]. L. Zhang, K. Zhao, H. Li, T. Zhang, D. Liu, Y. Han, J. Polym. Sci. Part B: Polym. Phys. 2019, 57, 1572.
[89]. H. Yuk, B. Lu, Z. X. H. bioelectronics, Chem. Soc. Rev. 2019, 48, 1642.
[90]. H. Shi, C. Liu, Q. Jiang, J. Xu, Adv. Electron. Mater. 2015, 1, 1500017.
[91]. J. Leal, S. Shaner, L. Matter, C. BOhler, M. Asplund, Adv. Mater. Interfaces 2023, 10, 2202041.
[92]. D. Won, J. Kim, J. Choi, H. Kim, S. Han, I. Ha, J. Bang, K. K. Kim, Y. Lee, T.-S. Kim, J.-H. Park, C.-Y. Kim, S. H. Ko, Sci. Adv. 2022, 8, eabo3209.
[93]. B. Lu, H. Yuk, S. Lin, N. Jian, K. Qu, J. Xu, X. Zhao, Nat. Commun. 2019, 10, 1043.
[94]. H. Yuk, B. Lu, S. Lin, K. Qu, J. Xu, J. Luo, X. Zhao, Nat. Commun. 2020, 11, 1604.
[95]. Y. Lu, C. Kacica, S. Bansal, L. M. Santino, S. Acharya, J. Hu, C. Izima, K. Chrulski, Y. Diao, H. Wang, H. Yang, P. Biswas, J. Schaefer, J. M. D'Arcy, ACS Appl. Mater. Interfaces 2019, 11, 47320.
[96]. Y. Lu, H. Yang, Y. Diao, H. Wang, C. Izima, I. Jones, R. Woon, K. Chrulski, J. M. D'Arcy, ACS Appl. Mater. Interfaces 2023, 15, 3433.
[97]. M. W. Lee, D.-J. Kwon, J. Park, J.-C. Pyun, Y.-J. Kim, H. S. Ahn, Chem. Commun. 2020, 56, 9624.
[98]. Y.-Y. Lee, H.o-Y. Kang, S. H. Gwon, G. M. Choi, S.-M. Lim, J.-Y. Sun, Y.-C. Joo, Adv. Mater. 2016, 28, 1636.
[99]. D. N. Heo, S.-J. Lee, R. Timsina, X. Qiu, N. J. Castro, L. G. Zhang, Mater. Sci. Eng., C 2019, 99, 582.
[100]. P. Jastrzebska-Perfect, G. D. Spyropoulos, C. Cea, Z. Zhao, O. J. Rauhala, A. Viswanathan, S. A. Sheth, J. N. Gelinas, D. Khodagholy, Sci. Adv. 2020, 6, eaaz6767.
[101]. S. B. Rauer, D. J. Bell, P. Jain, K. Rahimi, D. Felder, J. Linkhorst, M. Wessling, Adv. Mater. Technol. 2022, 7, 2100836.
[102]. S.-M. Kim, C.-H. Kim, Y. Kim, N. Kim, W.-J. Lee, E.-H. Lee, D. Kim, S. Park, K. Lee, J. Rivnay, M.-H. Yoon, Nat. Commun. 2018, 9, 3858.
[103]. Y. Kim, H. Noh, B. D. Paulsen, J. Kim, I.l-Y. Jo, H. Ahn, J. Rivnay, M.-H. Yoon, Adv. Mater. 2021, 33, 2007550.
[104]. N. Kim, S. Kee, S. H. o Lee, B. H. Lee, Y. H. o Kahng, Y.-R. Jo, B.-J. Kim, K. Lee, Adv. Mater. 2014, 26, 2268.
[105]. S. K. M. Jönsson, J. Birgerson, X. Crispin, G. Greczynski, W. Osikowicz, A. W. Denier van der Gon, W. R. Salaneck, M. Fahlman, Synth. Met. 2003, 139, 1.
[106]. L. Bießmann, N. Saxena, N. Hohn, M. A. Hossain, J. G. C. Veinot, P. Müller-Buschbaum, Adv. Electron. Mater. 2019, 5, 1800654.
[107]. A. Hu, L. Tan, X. Hu, L. Hu, Q. Ai, X. Meng, L. Chen, Y. Chen, J. Mater. Chem. C 2017, 5, 382.
[108]. J. W. Choi, M. G. Han, S. Y. Kim, S. G. Oh, S. S. Im, Synth. Met. 2004, 141, 293.
[109]. G. Cao, S. Cai, Y. Chen, D. Zhou, H. Zhang, Y. Tian, Polymer 2022, 252, 124952.
[110]. H. Zheng, Y. Jiang, J. Xu, Y. Yang, J. Wuhan Univ. Technol-Mat. Sci. Ed. 2011, 26, 422.
[111]. N. Paradee, A. Sirivat, Polym. Int. 2014, 63, 106.
[112]. Y. Lei, H. Oohata, S.-i Kuroda, S. Sasaki, T. Yamamoto, Synth. Met. 2005, 149, 211.
[113]. C. Wang, T. Yokota, T. Someya, Chem. Rev. 2021, 121, 2109.
[114]. A. O. Borode, N. A. Ahmed, P. A. Olubambi, Phys. Fluids 2019, 31, 071301.
[115]. C. Backes, A. M. Abdelkader, C. Alonso, A. Andrieux-Ledier, R. Arenal, J. Azpeitia, N. Balakrishnan, L. Banszerus, J. Barjon, R. Bartali, S. Bellani, C. Berger, R. Berger, M. M. B. Ortega, C. Bernard, P. H. Beton, A. Beyer, A. Bianco, P. Bøggild, F. Bonaccorso, G. B. Barin, C. Botas, R. A. Bueno, D. Carriazo, A. Castellanos-Gomez, M. Christian, A. Ciesielski, T. Ciuk, M. T. Cole, J. Coleman, et al., 2D Mater. 2020, 7, 022001.
[116]. H. Li, Y. Qiu, R. Soc. Open Sci. 2019, 6, 190241.
[117]. A. A. Markus, J. R. Parsons, E. W. M. Roex, P. de Voogt, R. Laane, Sci. Total Environ. 2015, 506-507, 323.
[118]. S. Yang, L. Jang, S. Kim, J. Yang, K. Yang, S.-W. Cho, J. Y. Lee, Macromol. Biosci. 2016, 16, 1653.
[119]. I. Perkucin, K. S. K. Lau, T. Chen, S. N. Iwasa, H. E. Naguib, C. M. Morshead, Adv. Healthcare Mater. 2022, 11, 2201164.
[120]. Y. Li, X. Liu, Q. Gong, Z. Xia, Y. Yang, C. Chen, C. Qian, Int. J. Biol. Macromol. 2021, 172, 41.
[121]. J.-H. Ding, H.-R. Zhao, H.-B. Yu, Sci. Rep. 2018, 8, 5567.
[122]. R. L. Keate, J. Tropp, C. P. Collins, H. O. T. Ware, A. J. Petty, G. A. Ameer, C. Sun, J. Rivnay, Macromol. Biosci. 2022, 22, 2200103.
[123]. D. M. y. Nguyen, Y. Wu, A. Nolin, C.-Y. Lo, T. Guo, C. Dhong, D. C. Martin, L. V. Kayser, Adv. Eng. Mater. 2022, 24, 2200280.
[124]. M. L. Clingerman, J. A. King, K. H. Schulz, J. D. Meyers, J. Appl. Polym. Sci. 2002, 83, 1341.
[125]. J. Li, J.-K. Kim, Compos. Sci. Technol. 2007, 67, 2114.
[126]. D. Stauffer, A. Aharony, Introduction To Percolation Theory, Taylor & Francis, London 1994.
[127]. C. W. Nan, Y. Shen, J. Ma, Annu. Rev. Mater. Res. 2010, 40, 131.
[128]. B. A. Praveena, N. Lokesh, A. Buradi, N. Santhosh, B. L. Praveena, R. Vignesh, Mater. Today Proc. 2022, 52, 1309.
[129]. M. Cianchetti, C. Laschi, A. Menciassi, P. Dario, Nat. Rev. Mater. 2018, 3, 143.
[130]. R. S. Jordan, Y. Wang, J. Polym. Sci. Part B: Polym. Phys. 2019, 57, 1592.
[131]. Y. Zheng, Y. Wang, F. Zhang, S. Zhang, K. D. Piatkevich, N. Zhou, J. K. Pokorski, Adv. Mater. Technol. 2022, 7, 2101514.
[132]. N. Lopez-Larrea, M. Criado-Gonzalez, A. Dominguez-Alfaro, N. Alegret, I. D. Agua, B. Marchiori, D. Mecerreyes, ACS Appl. Polym. Mater. 2022, 4, 6749.
[133]. M. Criado-Gonzalez, A. Dominguez-Alfaro, N. Lopez-Larrea, N. Alegret, D. Mecerreyes, ACS Appl. Polym. Mater. 2021, 3, 2865.
[134]. R. Pugliese, B. Beltrami, S. Regondi, C. Lunetta, Ann. 3D Print. Med. 2021, 2, 100011.
[135]. C. Yu, J. Schimelman, P. Wang, K. L. Miller, X. Ma, S. You, J. Guan, B. Sun, W. Zhu, S. Chen, Chem. Rev. 2020, 120, 10695.
[136]. R. van Lith, E. Baker, H. Ware, J. Yang, A. C. Farsheed, C. Sun, G. Ameer, Adv. Mater. Technol. 2016, 1, 1600138.
[137]. H. O. T. Ware, A. C. Farsheed, B. Akar, C. Duan, X. Chen, G. Ameer, C. Sun, Mater. Today Chem. 2018, 7, 25.
[138]. V. R. Feig, S. Santhanam, K. W. u McConnell, K. Liu, M. Azadian, L. G. Brunel, Z. Huang, H. Tran, P. M. George, Z. Bao, Adv. Mater. Technol. 2021, 6, 2100162.
[139]. Y. Ding, R. Fu, C. P. Collins, S.-F. Yoda, C. Sun, G. A. Ameer, Adv. Healthcare Mater. 2022, 11, 2201955.
[140]. S. Atifi, M.-N. Mirvakili, W. Y. Hamad, ACS Appl. Polym. Mater. 2022, 4, 5626.
[141]. J. Tropp, A. S. Mehta, X. Ji, A. Surendran, R. Wu, E. A. Schafer, M. M. Reddy, S. P. Patel, A. J. Petty, J. Rivnay, Chem. Mater. 2023, 35, 41.
[142]. K. Snigdha, B. K. Singh, A. S. Mehta, R. P. Tewari, P. K. Dutta, Int. J. Biol. Macromol. 2016, 93, 1639.
[143]. A. S. Mehta, B. K. Singh, N. Singh, D. Archana, K. Snigdha, R. Harniman, S. S. Rahatekar, R. p. Tewari, P. k. Dutta, J. Biomater. Appl. 2014, 29, 1314.

What is claimed is:

1. A conductive nanomaterial, comprising:
acid-crystalized poly(3,4-ethylenedioxythiophene) poly(styrenesulfonate) (PEDOT:PSS) nanoparticles (ncrys-PEDOT_{X}) with intrinsic dispersibility, wherein X represents an amount of an acid in a coagulation bath, and the amount is between 0 and 100%.

2. The conductive nanomaterial of claim 1, wherein the intrinsic dispersibility of the ncrys-PEDOT_{X} enables its homogeneous incorporation at desired loadings within diverse aqueous biomaterial solutions without additives or surfactants.

3. The conductive nanomaterial of claim 2, wherein the desired loadings is between 0-100%, preferably, varying within 0-5%, 5-10%, 10-20%, . . . up to 99%.

4. The conductive nanomaterial of claim 1, wherein the ncrys-PEDOT_{X} is directly incorporable within a hydrogel, a scaffold, a film, a hydrophobic polymer, an elastomer, thermoplastic, thermoset and/or an aqueous resin formation without sonication or external surfactants.

5. The conductive nanomaterial of claim 1, wherein the ncrys-PEDOT_{X} is biocompatible, and hemocompatible.

6. The conductive nanomaterial of claim 1, wherein the ncrys-PEDOT_{X} is usable in scalable, conductive, biocompatible, and modular platforms for bioelectronic applications.

7. The conductive nanomaterial of claim 1, wherein the ncrys-PEDOT_{X} is synthesized with an acid-based nonsolvent induced phase separation (NIPS) method by coagulating a PEDOT:PSS solution into stable aggregates of concentrated PEDOT with tuneable PSS surfactant.

8. The conductive nanomaterial of claim 7, wherein the PEDOT:PSS solution comprises PEDOT:PSS added in the coagulation bath including the acid in isopropanol (IPA).

9. The conductive nanomaterial of claim 8, wherein the acid comprises a sulfuric acid.

10. The conductive nanomaterial of claim 8, wherein increasing the concentrations of the acid within the coagulation bath correlates with increasing a PEDOT/PSS ratio.

11. The conductive nanomaterial of claim 10, wherein the degree to which PSS is removed and PEDOT crystallizes within the ncrys-PEDOT_{X} particles is directly related to the concentration of the acid within the coagulation bath.

12. The conductive nanomaterial of claim 10, wherein, by tuning a volume ratio of the acid within the coagulation bath, the PEDOT/PSS ratio in the ncrys-PEDOT_{X} is optimized to afford the ncrys-PEDOT_{X} with high conductivity (σ_{ncrys-PEDOT20}=410 S cm⁻¹) rivaling all the existing conjugated polymer particles.

13. The conductive nanomaterial of claim 10, wherein the ncrys-PEDOT_{X} has conductivities in a range of about 1-100 S cm⁻¹, about 100-400 S cm⁻¹, and/or about 400-800 S cm⁻¹.

14. The conductive nanomaterial of claim 13, wherein the ncrys-PEDOT₅ and ncrys-PEDOT₂₀ have conductivities of about 87 and 410 S cm⁻¹.

15. The conductive nanomaterial of claim 8, wherein crystallization of the ncrys-PEDOT_{X} particles enhances with increasing the concentration of the acid within the coagulation bath.

16. The conductive nanomaterial of claim 15, wherein a crystallite size of the ncrys-PEDOT_{X} particles increases from about 1.26 to 1.58 nm upon treatment with more concentrated sulfuric acid.

17. The conductive nanomaterial of claim 1, wherein the ncrys-PEDOT_{X} particles have no interfere with chemistries of crosslink hydrogels including hydrogen bonding, ionic bonding, Schiff-base chemistry, and radical photopolymerization.

18. The conductive nanomaterial of claim 1, wherein by directly adding the ncrys-PEDOT_{X} to a hydrogel formulation, a highly conductive composite is achieved with a percolation threshold between 0-5, 5-10, 10-15, or 15-20 wt % loading of the ncrys-PEDOT_{X}.

19. The conductive nanomaterial of claim 1, wherein the PEDOT:PSS is formed with one or more functional monomers to make the ncrys-PEDOT_{X} particles to have greater functionality.

20. A method of synthesizing a conductive nanomaterial, comprising:
dropwisely adding an aqueous solution of poly(3,4-ethylenedioxythiophene) poly(styrenesulfonate) (PEDOT:PSS) into a coagulation bath including an acid in isopropanol (IPA) to form a mixture thereof;
collecting particles from the mixture; and
subsequently comminuting the collected particles into a fine powder to form the conductive nanomaterial comprising acid-crystalized PEDOT:PSS nanoparticles (ncrys-PEDOT_{X}) with intrinsic dispersibility while maintaining high conductivity, wherein X represents an amount of the acid in the coagulation bath, and the amount is between 0 and 100%.

21. The method of claim 20, wherein the IPA is a nonsolvent that dehydrates the colloidal dispersion, enabling nonsolvent induced phase separation (NIPS).

22. The method of claim 20, wherein the acid comprises a sulfuric acid.

23. The method of claim 22, wherein the sulfuric acid is to stabilize segregation of insulating PSS from conductive PEDOT.

24. The method of claim 20, wherein conductivity enhancement from acid crystallization involves removal of PSS and enhanced crystallinity.

25. The method of claim 24, wherein increasing a concentration of the acid within the coagulation bath correlates with increasing a PEDOT/PSS ratio in the ncrys-PEDOT_{X}.

26. The method of claim 24, wherein the degree to which PSS is removed and PEDOT crystallizes within the ncrys-PEDOT_{X} particles is directly related to the concentration of the acid within the coagulation bath.

27. The method of claim 24, wherein, by tuning a volume ratio of the acid within the coagulation bath, the PEDOT/PSS ratio in the ncrys-PEDOT_{X} is optimized to afford the ncrys-PEDOT_{X} with high conductivity (σ_{ncrys-PEDOT20}=410 S cm⁻¹) rivaling all the existing conjugated polymer particles.

28. The method of claim 27, wherein the ncrys-PEDOT_{X} has conductivities in a range of about 1-100 S cm⁻¹, about 100-400 S cm⁻¹, and/or about 400-800 S cm⁻¹.

29. The method of claim 28, wherein the ncrys-PEDOT₅ and ncrys-PEDOT₂₀ have conductivities of about 87 and 410 S cm⁻¹.

30. The method of claim 20, wherein crystallization of the ncrys-PEDOT_{X} particles enhances with increasing the concentration of the acid within the coagulation bath.

31. The method of claim 30, wherein a crystallite size of the ncrys-PEDOT_{X} particles increases from about 1.26 to 1.58 nm upon treatment with more concentrated sulfuric acid.

32. A composite, comprising:
an aqueous biomaterial solution; and
acid-crystalized PEDOT:PSS nanoparticles (ncrys-PEDOT_{X}) loaded into the aqueous biomaterial solution, wherein X represents an amount of an acid in a coagulation bath, and the amount is between 0 and 100%,
wherein the ncrys-PEDOT_{X} acts as a filler with the highest conductivity of the particles while maintaining dispersibility.

33. The composite of claim 32, wherein the aqueous biomaterial solution is devoid of additives or surfactants.

34. The composite of claim 32, wherein the ncrys-PEDOT_{X} is loaded into the aqueous biomaterial solution by vortexing, stirring, shaking, centrifugation, milling, and/or the likes.

35. The composite of claim 32, wherein the aqueous biomaterial solution comprises a hydrogel synthesized from aqueous solutions of hydrophilic natural biopolymers including alginate, gelatin, collagen, and/or chitosan, or synthetic polymer formulations including pHEMA, PEO, and/or PEGDA.

36. The composite of claim 35, wherein the composite has a significant increase in conductivity with a percolation threshold between 0-5, 5-10, 10-15, or 15-20 wt % loading of the ncrys-PEDOT_{X}, with the highest loading displaying a remarkable conductivity of 1.1 S cm⁻¹.

37. The composite of claim 35, wherein the composite has about 8.2 S cm-1 with 5% particle loading, which is notably >400 fold higher than an EDOT postpolymerization in PEDGA alone (0.02 S cm⁻¹).

38. The composite of claim 32, wherein the ncrys-PEDOT_{X} has not interfered with chemistries of the aqueous biomaterial solution.

39. The composite of claim 32, wherein the ncrys-PEDOT_{X} has no deleterious impact on the stability of the aqueous biomaterial solution.

40. The composite of claim 32, wherein the composite comprises an aqueous photoprintable conductive resin is formulated through the addition of the ncrys-PEDOT_{X} into a mixture of poly(ethylene glycol)diacrylate (PEGDA) and lithium phenyl (2,4,6-trimethylbenzoyl) phosphinate (LAP) photoinitiator in water.

41. The composite of claim 40, wherein the aqueous PEGDA resin is utilized for 3D printing of a soft matter with complex form factors with high fidelity complex biomedical structures for biomedical applications.

42. The composite of claim 32, wherein the composite has high cell viability (>95%) at all loadings up to about 15% of ncrys-PEDOT_{X}.

43. The composite of claim 32, wherein the composite is biocompatible and/or hemocompatibible.
